(19) United States
(12) Patent Application Publication
(10) Pub. No.: US 2024/0402273 A1
(43) Pub. Date: Dec. 5, 2024

(54) INFECTION DIAGNOSIS AND CHARACTERIZATION USING DIFFUSION AND RELAXATION EDITED PROTON NMR SPECTROSCOPY

(71) Applicants: Bruker BioSpin GmbH, Ettlingen (DE); Murdoch University, Perth (AU)

(72) Inventors: Jeremy K. NICHOLSON, City Beach (AU); Elaine HOLMES, City Beach (AU); Samantha LODGE, Baldivis (AU); Julien WIST, City Beach (AU); Wolfgang BERMEL, Karlsbad (DE); Tony REINSPERGER, Karlsruhe (DE); Philipp NITSCHKE, City Beach (AU)

(21) Appl. No.: 18/262,262

(22) PCT Filed: Jan. 24, 2022
(86) PCT No.: PCT/IB2022/050593

§ 371 Date: Jul. 20, 2023

Related U.S. Application Data
(60) Provisional application No. 63/145,155, filed on Feb. 3, 2021.
(60) Provisional application No. 63/140,732, filed on Jan. 22, 2021.

(51) Int. Cl. G01R 33/465; G01N 33/50; G01R 33/46

(52) U.S. Cl. CPC G01R 33/465; G01N 33/5091; G01R 33/4616; G01R 33/4625; G01N 2333/775; G01N 2800/26; G01N 2800/32

(57) ABSTRACT

1H-NMR spectroscopic molecular markers are provided for identifying medical risk signatures such as SARS-CoV-2 infection, acute inflammation, or a cardiovascular risk condition. The markers use a combination of NMR intensity signals, including a Glyc signal from at least one N-acetyl (—NCOCH₃) glycoprotein and an SPC signal from a choline head group (⁺N—(CH₃)₃) of a supramolecular phospholipids cluster (SPC) present in HDL and LDL lipoprotein subfractions. The Glyc signal is in a chemical shift region from #=2.00 ppm to #=2.20 ppm, and includes signals GlycA (2.00 ppm to 2.09 ppm) and GlycB (2.09 ppm to 2.2 ppm). The SPC signal is in a chemical shift region from #=3.20 ppm to #=3.30 ppm, and includes signals SPC₁ (3.2 ppm to 3.235) ppm, SPC₂ (3.235 ppm to 3.26 ppm), and SPC₃ (3.26 ppm to 3.3 ppm). A system for identifying the markers is also provided.

### BACKGROUND OF THE INVENTION

### Field of the Invention

This invention relates generally to the field of nuclear magnetic resonance (NMR) based metabolic phenotyping and, more specifically, to the use of such a method for identifying and characterizing SARS-CoV-2 infections, inflammatory conditions and cardiovascular risks.

### Description of the Related Art

The COVID-19 disease pandemic resulting from SARS-CoV-2 infection has, so far, resulted in over 300 million cases and more than five million deaths worldwide. The range of clinical expression of COVID-19 is extreme, varying from asymptomatic or mild to severe respiratory distress and multiple organ damage, with or without respiratory involvement. There is an unmet need for accurate diagnosis and prediction of disease severity at an early stage so that individual infections can be monitored and managed effectively. There is also an unmet medical need for new functional markers of patient recovery in COVID-19, especially for the complex systemic complications of the disease and to monitor changes in risk level during and after the acute phase.

A metabolic phenoconversion approach was recently proposed to explore the systemic shifts in plasma biochemistry resulting from SARS-CoV-2 infection and the accompanying multi-system pathological disruptions caused by the virus. Phenoconversion for COVID-19 is associated with a range of metabolic biomarkers (lipoproteins, glycoproteins, amino acids, lipids and other metabolites) that can be derived from NMR spectroscopic and mass spectrometric data. Indeed, combining NMR and mass spectrometry (MS)-generated metabolic features into an integrated supervised classification model allowed excellent discrimination between SARS-CoV-2 positive subjects and controls. This approach also enabled deep insights to be gained into the systemic nature of the COVID-19 disease, with its distinctive embedded biomarker features, including those previously observed in diabetes, cardiovascular disease, liver dysfunction, neurological disruption and acute inflammation.

Proton NMR spectroscopy has been shown to be highly effective in detecting disease signatures in biofluids such as blood plasma, and multiple NMR methods have been applied to extract latent biomarker information either using physical NMR experiments including two dimensional methods or statistical spectroscopic methods such as Statistical Total Correlation SpectroscopY (STOCSY) and related techniques. Although physical procedures can be used to extract, separate and augment detection and identification of metabolites and lipoproteins in plasma, one of the key advantages of NMR spectroscopy is its non-invasive and non-destructive nature which enables the interrogation of molecular interactions complexation and physical dynamics of complex mixtures that can carry extra diagnostic information. This also allows the sample to be retained for further experimentation and elucidation of additional diagnostic information.

Plasma glycoproteins are biosynthesised and released mainly from the liver; they are enzymatically glycosylated and assist solubilization of multiple hydrophobic compounds in the blood. It has been reported that the well-resolved N-acetyl signals from glycosylated amino sugar residues in acute phase reactive proteins such as α-1 N-acetyl-glycoprotein in NMR spectra of blood plasma are elevated in multiple inflammatory states, including obesity, diabetes, cardiovascular disease, rheumatoid arthritis and systemic immune-pathological conditions such as HIV infection and systemic lupus erythematosus. These NMR signals are now widely described as GlycA and GlycB.

The GlycA signal (δ 2.03) is a composite of signals from primarily five proteins: α-1-acid glycoprotein, α-1-antichymotrypsin, α-1-antitrypsin, haptoglobin and transferrin. In a-1-acid glycoprotein the signal originates from five N-linked oligosaccharide chains on a backbone of 183 amino acid residues and is present at approximately 20 μM in healthy individuals. The α-1-acid glycoprotein has the strongest correlation with the GlycA signal and is thought to account for most of the signal, although inter-individual differences in the levels of these five glycoproteins have been reported. Multiple biological functions have been ascribed to α-1-acid glycoprotein, including modulating immunological function via a macrophage-released inhibitory factor that acts to prevent IL-1 activation of thymocyte proliferation, stimulating lymphocyte proliferation, serving as drug transporters and inhibiting platelet aggregation. Acute phase inflammation has been associated with two-to five-fold increases in plasma GlycA signals. The GlycB acetyl signal (δ 2.07) arising from glycoprotein N-acetylneuraminidino-groups have also been observed to increase in various inflammatory conditions such as diabetes and obesity. Both GlycA and GlycB have been shown to correlate with C-Reactive Protein (CRP) levels in plasma and it has been suggested that GlycA and GlycB may be superior biomarkers of systemic inflammation over CRP, the main clinical chemistry marker of inflammation. It has also been recently reported that GlycA and GlycB are significantly elevated in COVID-19 patients and are strong markers of disease positivity.

Low-density lipoprotein cholesterol (LDL), high-density lipoprotein cholesterol (HDL), apolipoprotein B100 (ApoB) and apolipoprotein A1 (ApoA1) have been associated with cardiovascular risks. In particular, the ApoB/AboA1 ratio has been shown to predict cardiovascular events. As each non-HDL particle carries one ApoB, non-HDL and non HDL-C have been reported to correlate with ApoB and may also be used as a marker for cardiovascular diseases. Although both parameters are correlated, their concordance is in discussion.

The non-destructive nature of NMR allows the study of complex supramolecular structures in the natural state in multiphasic samples such as blood plasma. The proton T₂ relaxation properties allow for differential spectral editing, for instance, to remove broad macromolecular envelopes in blood or plasma based on their short proton T₂'s. Translational diffusion can also be measured using pulsed field gradients and used to selectively attenuate signals from small molecules that have fast translational motion. Then, mathematical transformations allow the construction of 2D spectra as in Diffusion Ordered SpectroscopY (DOSY), which is now the commonly applied method in most fields. It is also possible to combine motional-editing in two-dimensional experiments such as Diffusion-Edited Total Correlation SpectroscopY (DE-TOCSY), or both types of motional editing together including Diffusion and Relaxation Editing (DIRE). It has previously been shown that DIRE spectra enhance signals from molecules with slow translational diffusion but with high segmental motional freedom, and these requirements are satisfied by plasma glycoproteins and molecules constrained within certain lipoprotein sub-compartments.

### SUMMARY OF THE INVENTION

In accordance with the present invention, Nuclear Magnetic Resonance (NMR) spectroscopy based metabolic phenotyping of plasma is employed to reveal novel diagnostic molecular signatures of inflammation and/or other medical conditions, such as SARS-CoV-2 infection. The NMR methods may use a Dlffusional and Relaxation Editing (DIRE) pulse sequence, with or without additional relaxation delay, diffusion or scalar couplings editing, such as J-coupling editing (JEDI). Other pulse sequences may also be used, such as a Pulsed Gradient Spin Echo (PGSE) sequence, a Pulsed Gradient Double Echo (PGDE) sequence, or a Pulsed Gradient Spin Echo×5 (PGSE-5).

The NMR analysis can be done using plasma or serum samples from patients, and the features that are measured show clear differences between those patients who are comparatively healthy and those have the condition in question, such as SARS-CoV-2 RT-PCR positive respiratory patients. In particular, the NMR spectra produced show unique biomarker signal combinations and patterns conferred by differential concentrations of metabolites with selected molecular mobility properties. These include: a) composite N-acetyl (—NCOCH₃) signals from α-1-acid glycoprotein and other glycoproteins (GlycA and GlycB) that are elevated in SARS-CoV-2 positive patients (p=2.52×10⁻¹⁰ and 1.25×10⁻⁹ versus controls respectively); and b) newly-identified Supramolecular Phospholipid Composite signals from the —⁺N—(CH₃)₃ choline headgroups that are associated with HDL and LDL subfractions. In one embodiment, two such signals are considered: SPC-A, which corresponds to phospholipids in the HDL subfraction; and SPC-B, which corresponds to a phospholipid component of LDL. In another embodiment, the SPC signals correspond to three different regions of SPC peaks: SPC₃, which correlates with LDL (a signal range of δ 3.26-3.30 ppm); SPC₂, which correlates with HDL (a signal range of δ 3.235-3.26 ppm); and SPC₁, which correlates with H4PL (the subfraction 4 of HDL, i.e., the higher density fraction) (a signal range of δ 3.20-3.235).

The overall SPC signal is equal to the sum of the signals in the subdivided SPC regions. Thus, SPCₜₒₜₐₗ=SPC-A+SPC-B in one embodiment, and SPCₜₒₜₐₗ=SPC₁+SPC₂+SPC₃ in another embodiment. As a whole, SPC appears reduced in SARS-CoV-2 positive patients relative to both controls (p=1.40×10⁻⁷) and SARS-CoV-2 negative patients (p=4.52×10⁻⁸), but is not significantly different between controls and SARS CoV-2 negative patients. SPC/GlycA ratios are also significantly different for normal vs SARS-CoV-2 positive patients (p=1.23×10⁻¹⁰) and for SARS-CoV-2 negatives versus positives (p=1.60×10⁻⁹). By using SPCₜₒₜₐₗ and SPCₜₒₜₐₗ/GlycA as sensitive new molecular markers for diagnosing certain conditions, such as SARS-CoV-2 positivity, the invention augments current COVID-19 diagnostics and may be employed in functional assessment of the disease recovery process.

The collection of the biomarkers described above may be done in accordance with an exemplary embodiment of the invention as discussed below. Although the description is with regard to SARS CoV-2 infection, those skilled in the art will understand that this represents only an example of the conditions for which such biomarkers may be collected. The method is equally applicable to cardiovascular risk, inflammatory states, or other similar acute or chronic conditions. Moreover, those skilled in the art will understand that the invention is not limited to this particular sequence of steps, and that different variations may exist for collecting the relevant data.

Sample preparation—Blood is collected from SARS CoV-2 positive and matched SARS CoV-2 negative subjects using standard phlebotomy methods. The collection tube is incubated and spun at a temperature and speed that meets known guidelines. The plasma or serum is removed from the blood collection tubes and centrifuged to obtain a supernatant, to which is added an appropriate buffer. It is then transferred to an NMR tube, which is placed in the NMR instrument for analysis.

NMR verification check—prior to analysis the NMR spectrometer should be calibrated using calibration samples, such as a temperature calibration sample, a sucrose sample and a Quantref sample or a similar method that ensures reproducible quantitative data. A spectrum of the plasma/serum sample is then acquired using the desired pulse sequence, such as a DIRE sequence. Although 1D NMR spectra are usually quite complicated with thousands of peaks, the DIRE NMR experiment edits out many of these peaks leaving only those from a flexible domain of macromolecules such as proteins and large phospholipid complexes. The NMR measurement is then carried out on the blood plasma or serum sample and the obtained NMR signal intensities are used for data analysis.

Data analysis—Prior to statistical analysis, all spectra are pre-processed. In the exemplary embodiment, the residual water resonances are removed (δ 4.5-5.0), as are the chemical shift regions where no signals of interest are observed (δ<0.25 and δ>9.5). The anomeric glucose peak is set to δ5.23 ppm. In this document, all the chemical shifts were reported after calibration to Glucose. Optionally, the spectra are then baseline corrected and calibrated, although doing so is not mandatory.

The statistical analysis is performed using a multivariate statistical procedure such as O-PLS-DA, which produces a scores plot in which each point corresponds to a sample and gives a maximum separation between samples of the different classes. The model also provides loadings values which correspond to the various NMR intensities, which are examined to determine which NMR spectral features are responsible for the separation seen in the scores' plot. This operation is done using standard statistics to ensure that the derived features are statistically valid. The NMR features responsible for class separation are then examined.

An initial visual inspection of the scores plot is done to determine, if possible, specific molecules that are responsible for the features. More specialized NMR experiments are thereafter used, as necessary, to provide information on the class separating molecules (the biomarkers), which leads to identification on a molecular basis. These include statistical approaches such as STOCSY, the use of appropriate databases, and a range of 1- and 2-dimensional NMR spectra. The derived classifying NMR features can be tested using spectra from further samples (a test set) to check the validity of the class prediction. This test set can be a sub-set of the original spectral set or new spectra.

In order to get the intensity of the markers SPCₜₒₜₐₗ (δ 3.20-3.30; Glucose 5.23), GlycA (δ2.03; Glucose 5.23) and GlycB (δ2.07; Glucose 5.23), the points for each of these regions and for each sample are summed and used to calculate the certain informative ratios, such as SPCₜₒₜₐₗ/GlycA. If the intensity values or ratios are below (or above) a given threshold, it can be deduced that the patient has a condition, especially an inflammatory or risk signature, such as a SARS-CoV-2 positivity.

In the context of this description, NMR signal intensity shall refer to the peak maximum height or, more preferably, the peak integrals which correspond to the area under the peak, which is typically more accurate as it remains constant if the peak shape varies, e.g., due to relaxation differences.

The addition of J-coupling editing (JEDI) to the DIRE experiment improves the accuracy of GlycA and GlycB measurements, using the same protocol for sample preparation and data processing, as it removes perturbations from lipoproteins and lipids in the vicinity of the GlycA peak. The correlation between GlycA and GlycB values obtained with JEDI and similar sequences (that include J-coupling editing) is much higher than the ones obtained using DIRE only, as the interference from the lipoproteins is efficiently suppressed by JEDI.

SPC is associated with both HDL and LDL fractions, primarily. More specifically, different regions of the SPC peaks can be defined (identified herein as SPC₁, SPC₂ and SPC₃) which are found to correlate with LDL (SPC₃ δ 3.26-3.30 ppm), HDL (SPC₂ δ 3.235-3.26 ppm) and H4PL (SPC₁ δ 3.20-3.235), and could be associated by linear regression. As the concentrations of LDL and HDL are related to the concentrations of apoprotein B-100 and A1, respectively, the SPC peak can be used as a marker of cardiovascular risk. The downfield region (left hand side) of the SPC peak stems mainly from LDL contributions, the center region of the peak is associated with the HDL fraction, while the highfield region (right hand side) is connected to the H4 subfraction.

In addition to the direct diagnostic value of the biomarker signals and ratios, a significant association is observed between the SPC/GlycA ratio and BMI (Body Mass Index) intervals. This evidences the potential of the aforementioned SPC biomarkers in a wider range of applications than those discussed herein, as well and its complementarity with the GlycA biomarker for use in other diagnostics.

The present invention relates also to the use of Glyc and/or SPC (or any subfraction of them) as biomarkers for the diagnosis of SARS-CoV-2 and/or for the assessment of a functional recovery from a SARS-CoV-2 infection.

Furthermore, the present invention relates to a system for extracting related medical information referring to a SARS-CoV-2 condition or risk or inflammatory condition by proton NMR, the system comprising: an NMR spectrometer for acquiring at least one NMR spectrum of an in vitro blood plasma or serum sample; and a processor in communication with the NMR spectrometer. The processor is configured to obtain concentration measurements of the biomarkers Glyc and SPC in said blood plasma or serum sample which are referred to NMR signals of Glyc (δ 2.00-2.20 ppm) and the choline head group (+N—(CH3)3) signal at δ 3.20-3.30 ppm from phospholipids (=SPC signal), and (ii) calculate an inflammatory condition based on the obtained signal intensities and their ratios.

The invention thus provides 1H-NMR spectroscopic molecular markers and the use thereof for identifying one or more medical risk signatures in an in vitro blood plasma or serum sample from a patient by 1H NMR spectroscopy. In general, the markers comprise a combination of NMR intensity signals having magnitudes that are significantly different from known corresponding NMR intensity levels for a healthy patient, including a Glyc signal from at least one N-acetyl (—NCOCH₃) glycoprotein and an SPC signal from a choline head group (⁺N—(CH₃)₃) of a supramolecular phospholipids cluster (SPC) present in HDL and LDL lipoprotein subfractions. In the exemplary embodiment, the Glyc signal is in a chemical shift region from δ=2.00 ppm to δ=2.20 ppm and the SPC signal is in a chemical shift region from δ=3.20 ppm to δ=3.30 ppm. The Glyc signal itself may be subdivided into a signal GlycA in a chemical shift subregion of δ=2.00 ppm to δ=2.09 ppm and a signal GlycB in a chemical shift subregion of δ=2.09 ppm to δ=2.2 ppm. Similarly, SPC signal may be subdivided into a signal SPC, in a chemical shift subregion from δ=3.2 ppm to δ=3.235 ppm, a signal SPC₂ in a chemical shift subregion from δ=3.235 ppm to 3.26 ppm, and a signal SPC₃ in a chemical shift subregion from δ=3.26 ppm to δ=3.3 ppm.

The molecular markers may also include various ratios of the Glyc and SPC signal or portions thereof, such as a ratio of NMR peak intensities of Glyc or either of GlycA or GlycB to NMR peak intensities of SPC or one or more of SPC₁, SPC₂ or SPC₃. For example, one useful ratio is SPCₜₒₜₐₗ/GlycA, where SPCₜₒₜₐₗ=SPC₁+SPC₂+SPC₃. The markers are highly useful, for example, for diagnosing medical risk signatures that include a SARS-CoV-2 infection, acute inflammation, certain cardiovascular risk conditions, as well as for assessment of a functional recovery from a SARS-CoV-2 infection. To use the markers for diagnosing such a risk signature, a blood plasma or serum sample is obtained from a patient, and an NMR measurement of the sample is performed to obtain a spectrum of NMR intensities. The magnitudes of a combination of the Glyc and SPC NMR intensity signals are then determined, and the presence of the risk signature is diagnosed when the magnitudes of the glycoprotein NMR intensities and the SPC NMR intensities, or the ratios derived therefrom, are significantly different from known corresponding NMR intensity levels or ratios for a healthy patient, such as by being beyond a predetermined threshold.

A system for identifying the medical risk signatures is also provided by the invention, and includes an NMR spectrometer for acquiring at least one 1H NMR spectrum of the vitro blood plasma or serum sample, and a data processor in communication with the NMR spectrometer. The data processor is configured to obtain concentration measurements of the NMR intensity signals of one or more of the markers, which would indicate the presence of the disease condition when their magnitudes are significantly different from known corresponding NMR intensity levels for a healthy patient. As discussed above, the markers include a Glyc signal from at least one N-acetyl (—NCOCH₃) glycoprotein and an SPC signal from a choline head group (⁺N—(CH₃)₃) of a supramolecular phospholipids cluster (SPC) present in HDL and LDL lipoprotein subfractions.

### BRIEF DESCRIPTION OF THE DRAWINGS

FIG. 1A is a 600 MHz ¹H NMR plot showing a standard water suppressed single pulse NMR spectrum of a plasma sample from a typical SARS-CoV-2 positive patient.

FIG. 1B shows the water suppressed CPMG spin-echo spectrum of the sample used in the plot of FIG. 1A.

FIG. 1C shows the DIRE spectrum of the sample used in the plot of FIG. 1A.

FIG. 1D shows the DIRE spectrum of the plasma from a representative healthy control individual.

FIG. 2A shows a PCA of the DIRE spectra of healthy controls, SARS-CoV-2 positive patients, SARS-CoV-2 negative non-hospitalised patients, SARS-CoV-2 negative hospitalised patients and serology (+) participants.

FIG. 2B shows the loadings of principal component 1 of FIG. 2A.

FIG. 2C shows the loadings of principal component 2 of FIG. 2A.

FIG. 2D is a DIRE OPLS-DA plot showing the training model consisting of SARS-CoV-2 positive patients (n=7), age and sex matched to the healthy control group (n=7).

FIG. 2E shows OPLS-DA loadings of the training model.

FIG. 2F shows an OPLS-DA plot showing the training model with the healthy control samples (n=19) projected into the model.

FIG. 2G is a DIRE OPLS-DA plot showing the training model with the SARS-CoV-2 positive patients (n=51 samples) projected into the model.

FIG. 2H is a DIRE OPLS-DA plot showing the training model with the SARS-CoV-2 negative patients (n=34) projected into the model.

FIG. 2I is a DIRE OPLS-DA plot showing the training model with the projected serology (+) participants (n=6).

FIG. 3A shows a 1D STOCSY of the full DIRE spectra using the SPC-A peak as the driver peak to determine the peaks highly correlated.

FIG. 3B shows an expanded region δ 2.5-5.5, indicating strong correlation of the driver peak (SPC-A peak) with peaks at δ 3.69 and 4.34.

FIG. 3C shows an expanded region δ 0.5-2.5, indicating strong correlation of the driver peak (SPC-A peak) with peaks at δ 0.8 and δ 1.2.

FIG. 3D shows a 1D STOCSY of the full DIRE spectra using the SPC-B peak as the driver peak to determine the peaks highly correlated.

FIG. 3E shows an expanded region δ 2.5-5.5, indicating strong correlation of the driver peak with peaks at δ 3.69 and 4.34.

FIG. 3F shows an expanded region δ 0.5-2.5, indicating strong correlation of the driver peak, SPC-B with peaks at δ 0.8 and δ 1.2.

FIG. 4A shows a known sequence designated DIRE-1.

FIG. 4B shows a modified DIRE pulse sequence used herein (designated DIRE).

FIG. 4C shows a first DIRE pulse sequence variation that uses a perfect echo instead of a regular spin echo to address J-modulation effects.

FIG. 4D shows a second DIRE pulse sequence variation in which the spin echo block acting as a T₂ filter and the LED diffusion block are separated.

FIG. 4E shows a third DIRE pulse sequence variation in which a combination of the pulse sequences of FIGS. 5C and 5D introduce the perfect echo and separation of T₂ filter from the LED diffusion block.

FIG. 4F shows the Pulsed Gradient Perfect Echo (PGPE) pulse sequence in accordance with the invention, highlighting the three editing approaches from JEDI; relaxation, diffusion and J-editing.

FIG. 4G shows a Pulsed Gradient Spin Echo (PGSE) pulse sequence in accordance with the invention.

FIG. 4H shows a Pulsed Gradient Double Echo (PGDE) pulse sequence in accordance with the invention.

FIG. 4I shows the Pulsed Gradient Spin Echo×5 (PGSE-5) pulse sequence in accordance with the invention.

FIG. 5 shows the GlycA peak integrals determined from the DIRE NMR spectra for a healthy cohort, SARS-CoV-2 positive and SARS-COV-2 negative patients.

FIG. 6 shows the GlycB peak integrals determined from the DIRE NMR spectra for same cohorts as FIG. 5.

FIG. 7A depicts the 1D STOCSY of the DIRE spectra using the peak at δ 2.03 (GlycA) as the driver peak to determine the peaks highly correlated.

FIG. 7B shows an expanded region δ 3.0-5.0, indicating strong correlation of the driver peak (GlycA) with peaks at δ 3.69 and δ 4.34.

FIG. 7C shows an expanded region δ 0.5-2.5, indicating strong correlation of the driver peak (GlycA) with peaks at δ 0.8 and δ 1.2.

FIG. 8A is an NMR plot of a ¹H¹³C edited Heteronuclear Single Quantum Coherence (HSQC) spectrum of a plasma sample.

FIG. 8B is an NMR plot of a ¹H¹³C Heteronuclear Multiple Bond Correlation (HMBC) from the same plasma sample as was used for FIG. 8A.

FIG. 9 is an NMR plot showing a diffusion edited (DE)-TOCSY sub-spectrum.

FIG. 10A is a stacked NMR plot showing presaturation and DIRE spectra for a 1,2-dilinoleoyl-sn-glycero-3-phophocholine standard in plasma, as well as the resulting DIRE difference spectrum.

FIG. 10B is a stacked NMR plot showing presaturation and DIRE spectra for a L-α-phophatidylcholine standard in plasma, as well as the resulting DIRE difference spectrum.

FIG. 10C is a stacked NMR plot showing presaturation and DIRE spectra for a 1,2-dipalmitoyl-sn-glycero-3-phosphocholine standard in plasma, as well as the resulting DIRE difference spectrum.

FIG. 10D is a stacked NMR plot showing presaturation and DIRE spectra for a L-α-lysophosphatidylcholine standard in plasma, as well as the resulting difference spectrum.

FIG. 11 depicts an NMR plot of ¹H 1D presat. And DIRE before and after titration of α₁-acid glycoprotein standard solution (16 μL of 9.6 mg α₁-acid glycoprotein in 70 μL NaCl solution), and the resulting DIRE difference spectrum. (bottom) showing the NMR signature of α₁-acid glycoprotein in plasma.

FIG. 12 shows a direct comparison of STOCSY to the DIRE-Difference spectrum of α-1-acid-glycoprotein titrated into plasma

FIG. 13 shows a direct comparison of STOCSY (top) with the SPC (δ 3.20-3.30) as the driver to the DIRE-Difference spectrum of 1,2-dilinoleoyl-sn-glycero-3-phosphocholine titrated into plasma (bottom). Perfect signal overlap is seen for both spectra.

FIG. 14A shows a spectrum that includes large molecules and small molecules, with a significant overlap and increased baseline from proteins, mainly albumin.

FIG. 14B is a spectrum in which a CPMG pulse sequence removes baseline contributions from the protein background, but not the overlapping peaks from small molecules and lipids.

FIG. 14C depicts a spectrum from a DIRE experiment that filters baseline and small molecules contributions making the measurement of SPC accessible.

FIG. 14D shows a spectrum in which JEDI-PGPE additional J-editing removes lipoprotein overlap from GlycA and GlycB.

FIG. 14E depicts a spectrum which shows that JEDI-PGSE gives similar results to the PGPE but gives rise to increased antiphase contributions in the baseline and residual lipid peaks.

FIG. 14F shows a spectrum that demonstrates how JEDI-PGDE perfectly removes any lipids interfering with GlycA and GlycB but suffers from a lower signal-to-noise ratio.

FIG. 15A depicts DIRE, JEDI-PGPE and selective TOCSY spectra of a first serum sample with medium lipoprotein concentration.

FIG. 15B depicts DIRE, JEDI-PGPE and selective TOCSY spectra of a second serum sample with medium lipoprotein concentration.

FIG. 16 depicts DIRE, JEDI-PGPE and selective TOCSY spectra of a third serum sample with high lipid concentration.

FIG. 17A shows an overlap of a DIRE spectrum, the corresponding selective TOCSY and a difference spectrum before and after addition of a 2 μL lipid mix of parenteral nutrition.

FIG. 17B shows the overlap of the JEDI-PGPE spectrum of a plasma sample before addition of a 2 μL lipid mix and the JEDI-PGPE difference spectrum before and after addition of the lipid mix; the difference spectrum yields the net lipid mix pattern in solution for the JEDI-PGPE.

FIG. 18A shows a JEDI-PGSE for a serum sample spiked with 2 μL of lipid mixture from parenteral nutrition.

FIG. 18B shows a JEDI-PGPE for a serum sample spiked with 2 μL of lipid mixture from parenteral nutrition.

FIG. 19A shows a spectrum for 1D ¹H with solvent suppression according to the invention.

FIG. 19B shows a CPMG spectrum according to the invention.

FIG. 19C shows a DIRE spectrum according to the invention.

FIG. 19D shows a JEDI-PGPE spectrum according to the invention.

FIG. 19E shows a JEDI-PGSE spectrum according to the invention.

FIG. 19F shows a JEDI-PGDE spectrum according to the invention.

FIG. 19G shows a PGSE-5 (G) spectrum according to the invention.

FIG. 20A shows a spectrum for 1D with solvent suppression according to the invention.

FIG. 20B shows a CPMG spectrum according to the invention.

FIG. 20C shows a DIRE spectrum according to the invention.

FIG. 20D shows a JEDI-PGPE spectrum according to the invention.

FIG. 20E shows a JEDI-PGSE spectrum according to the invention.

FIG. 20F shows a JEDI-PGDE spectrum according to the invention.

FIG. 20G shows a PGSE-5 spectrum according to the invention

FIG. 21A provides a JEDI-PGPE comparison of the high frequency region of Glyc for a serum and plasma sample of the same individual.

FIG. 21B shows a JEDI-PGPE high frequency region of Glyc for a plasma sample before and after addition of 5.2 mg fibrinogen.

FIG. 22A shows a linear fit for the relationship of GlyA and GlycB by the DIRE experiment.

FIG. 22B shows a linear fit for the relationship of GlyA and GlycB by the JEDI-PGPE experiment

FIG. 22C depicts box plots showing the GlycA integral for the longitudinal serum samples of pregnant women samples measured by DIRE and JEDI-PGPE (black).

FIG. 22D shows Z-scores indicating which samples changed their respective rank (“mis-classification”) for DIRE against JEDI. Strongest outliers are marked with an asterisk.

FIG. 23A shows a heatmap of the SPC spectral region of a control set using DIRE NMR experiments and selected plasma lipid components.

FIG. 23B shows a heatmap like that of FIG. 23A, but for SARS-CoV-2 infected patients.

FIG. 23C depicts the median DIRE spectra for samples from healthy patients and SARS-CoV-2 infected patients, respectively.

FIG. 24 plots the SPC/Glyc ratio for a control cohort, a SARS-CoV-2 positive cohort and the same cohort 3 months after the acute phase.

FIG. 25A shows the integrals of GlycA from a large, normal population cohort (n=2045) stratified into BMI intervals ranging from 16-60.

FIG. 25B shows the integrals of GlycB for the same cohort as FIG. 25A.

FIG. 25C shows the integrals of SPC for the same cohort as FIG. 25A.

FIG. 25D shows the integrals of the SPC/GlycA ratio for the same cohort as FIG. 25A.

FIG. 26 shows the general structure of a system according to the invention for identifying a medical risk signature, including an NMR spectrometer and a data processor.

FIG. 27 is a general flow diagram showing the steps of a diagnostic method according to the invention.

### DETAILED DESCRIPTION

The biomarkers and diagnostic methods using them are demonstrated herein, in part, by describing a research study conducted by the inventors that confirmed their effectiveness, in particular with regard to the diagnosis of SARS-CoV-2 infection. The following discussion of this study provides an exemplary embodiment for implementation of the invention, but those skilled in the art will recognize that the principles applied therein are extendable to other specific applications, which are likewise considered to be within the scope of the present invention.

Patient Enrolment and Sample Collection for Western Australian Cohort: Blood plasma samples were collected into potassium EDTA sample tubes from a cohort of adult individuals in a study initiated at the Fiona Stanley Hospital in the Western Australia South Metropolitan Health Service catchment as part of the International Severe Acute Respiratory and Emerging Infection Consortium (ISARIC)/World Health Organisation (WHO) pandemic trail framework (SMHS Research Governance Office PRN: 3976 and Murdoch University Ethics no. 2020/052). Healthy control participants were enrolled as volunteers, provided study details and written informed consent was obtained prior to data collection in accordance with ethical governance (Murdoch University Ethics no. 2020/053). Five groups of participants were recruited from the Fiona Stanley and Royal Perth Hospitals: i) patients who presented COVID-19 disease symptoms and subsequently tested positive for SARS-CoV-2 infection from upper and/or lower respiratory tract swabs by RT-PCR (n=17 patients, sampled at various times resulting in n=58 plasma specimens); ii) healthy controls who had not exhibited COVID-19 disease symptoms (n=26 participants); iii) individuals with respiratory disease symptoms and who tested negative for SARS-CoV-2 and were non-hospitalized (n=23 participants); iv) hospitalized SARS-CoV-2 negative respiratory patients (n=11); and v) individuals who were serologically IgA positive for COVID-19 (n=6). Serological testing for SARS-CoV-2 antibodies was performed at the PathWest clinical testing laboratories, Western Australia using 10 μL of plasma in a commercial point-of-care serological COVID-19 IgA/IgG test. Samples were considered as SARS-CoV-2 positive if IgA>1.0 or equivocal where IgA=0.8-1.0. Demographic data together with the clinical symptoms are shown below in Tables 1-3. IgA and IgG levels are reported in Table 4. Plasma samples were stored at −80° C. until required for analysis.

TABLE 1
Full cohort demographic data

| | | | | | SARS-CoV-2 | | SARS-CoV-2 | | | |
|---|---|---|---|---|---|---|---|---|---|---|
| | SARS-CoV-2 | | Healthy | | Negative (−) | | Negative (−) | | Serology IgA | |
| | Positive (+) | | Controls | | Hospitalized | | non-hospitalized | | positive | |
| | (N = 17) | | (N = 26) | | (N = 11) | | (N = 23) | | (N = 6) | |
| Sex, Male | 10 | (58.82%) | 15 | (57.69%) | 5 | (45.45%) | 11 | (47.83%) | 5 | (83.33%) |
| Age, years [SD] | 67.53 | [±11.07] | 52.19 | [±15.07] | 67.45 | [±21.03] | 49.00 | [±16.10] | 62.71 | [±18.49] |
| BMI, kg/m² [SD] | 35.26 | [±11.10] | 26.29 | [±3.41] | 25.73 | [±5.01] | 29.92 | [±7.86] | 26.17 | [±4.07] |

| | | | | | | | | | |
|---|---|---|---|---|---|---|---|---|---|
| Pre-diabetic/ | 3 | (17.65%) | 2 | (7.69%) | 1 | (4.54%) | 3 | (13.04%) | 0 |
| diabetic | | | | | | | | | |

| | | | | | | | | | |
|---|---|---|---|---|---|---|---|---|---|
| Hypertension | 8 | (47.06%) | 2 | (7.69%) | 0 | 4 | (17.39%) | 3 | (50.00%) |

| | | | | | | | |
|---|---|---|---|---|---|---|---|
| Asthma | 3 | (17.65%) | 0 | 0 | 6 | (26.08%) | 0 |

| | | | | | | | |
|---|---|---|---|---|---|---|---|
| COPD | 0 | 0 | 2 | (9.09%) | 1 | (4.35%) | 0 |

| Arthritis | 4 | (23.53%) | 0 | 0 | | 0 | 0 |
|---|---|---|---|---|---|---|---|
| Glaucoma | 2 | (11.76%) | 0 | 0 | | 0 | 0 |
| Dyslipidemia | 1 | (5.88%) | 0 | 0 | | 0 | 0 |
| Chronic Renal | 2 | (11.76%) | 0 | 3 | (13.64%) | 0 | 0 |
| Disease | | | | | | | |
| Chronic heart | 2 | (11.76%) | 0 | 2 | (9.09%) | 0 | 0 |
| disease | | | | | | | |

TABLE 2
Symptom presentation in SARS-CoV-2 positive patients

| | SARS-CoV-2 positive | |
|---|---|---|
| Symptom | patients (n = 17) | |
| Fever | 11 | (64.7%) |
| Cough | 9 | (52.9%) |
| Shortness of breath | 6 | (35.3%) |
| Sore throat | 1 | (5.9%) |
| Rhinorrhoea | 2 | (11.8%) |
| Wheeze | 1 | (5.9%) |
| Chest pain | 1 | (5.9%) |
| Myalgia | 2 | (11.8%) |
| Joint pain | 1 | (5.9%) |
| Fatigue | 5 | (29.4%) |
| Headache | 2 | (11.8%) |
| Confusion | 2 | (11.8%) |
| Abdominal pain | 1 | (5.9%) |
| Vomit | 2 | (11.8%) |
| Diarrhoea | 4 | (23.5%) |

| | |
|---|---|
| Conjunctivitis | 0 |
| Lymphadenopathy | 0 |

TABLE 3
Cohort demographic data for the training set

| | SARS-CoV-2 positive | | Healthy controls | |
|---|---|---|---|---|
| | patients (n = 7) | | (n = 7) | |
| Male sex | 3 | (42.86%) | 3 | (42.86%) |
| Age, years [SD] | 64.57 | [±13.13] | 65.00 | [±13.04] |
| BMI, kg/m² [SD] | 37.49 | [±10.56] | 26.91 | [±3.57] |
| Pre diabetic/diabetic | 1 | (14.29%) | 1 | (14.29%) |
| Hypertension | 4 | (57.14%) | 1 | (14.29%) |

| Asthma | 1 | (14.29%) | 0 |
|---|---|---|---|
| Chronic obstructive | 0 | | 0 |
| pulmonary disease | | | |
| Arthritis | 1 | (14.29%) | 0 |
| Glaucoma | 1 | (14.29%) | 0 |
| Dyslipidemia | 0 | | 0 |
| Chronic Renal Disease | 0 | | 0 |
| Chronic heart disease | 1 | (14.29%) | 0 |

TABLE 4
Clinical Serology evaluation of IgA and IgG
concentrations Signal to Cut-off ratio.

| Subject | IgA | IgG |
|---|---|---|
| 1 | 1.5 | 0.3 |
| 2 | 3 | 0.1 |
| 3 | 1.1 | 0.2 |
| 4 | 1.6 | 0.4 |
| 5 | 0.8 | 0 |
| 6 | 2.9 | 0.8 |
| 7 | 1.5 | 0.2 |

Patient Enrolment and Sample Collection for Autonomous Community of the Basque Country (Spain) cohort: The cohort consisted of i) patients who tested positive for SARS-CoV-2 infection from upper and/or lower respiratory tract swabs by RT-PCR (n=36) and ii) healthy control participants (n=80). All serum samples were collected by the Basque Biobank for research (BIOEF). Healthy serum samples were collected before the COVID-19 pandemic from the active population while the COVID-19 samples were collected at the Cruces University Hospital (Barakaldo, Spain) from patients who presented compatible symptoms, confirmed by a RT-PCR assay on nasal swab samples. All participants provided informed consent to clinical investigations, according to the Declaration of Helsinki, and all data were anonymized to protect their confidentiality. The sample handling protocol was evaluated and approved by the Comité de Ética de Investigación con medicamentos de Euskadi (CEIm-E, PI+CES-BIOEF 2020-04 and PI219130). Shipment of human samples to ANPC had the approval of the Ministry of Health of the Spanish Government. Samples were stored at −80° C.

Patient Enrolment and Sample Collection for Microbiome Understanding in Maternity Study (MUMS) cohort: The cohort consisted of women (n=99) who were recruited in their first trimester of pregnancy and followed through at seven time points: trimesters; one, two and three, the time of birth and then six weeks, six months and 12 months postpartum. All serum samples were collected at the University of New South Wales (UNSW), Microbiome Research Centre (MRC), Sydney, Australia. All participants provided informed consent to clinical investigations, according to the Declaration of Helsinki, and all data were anonymized to protect their confidentiality. The sample handling protocol was evaluated and approved by the South Eastern Sydney Local Health District Research Ethics Committee (17/293 (HREC/17/POWH/605)). Shipment of human samples to ANPC had the approval of the University of New South Wales. Samples were stored at −80° C.

Sample Processing: Blood samples were centrifuged at 13,000 g to separate the plasma, which was then frozen at −80° C. until use. Plasma samples were thawed at 20° C. for 30 minutes then centrifuged at 13,000 g for 10 minutes at 4° C. Plasma samples were prepared in 5 mm outer diameter SampleJet™ NMR tubes, following the recommended procedures for in vitro analytical and diagnostics procedures using 300 μL of plasma mixed with 300 μL of phosphate buffer (75 mM Na₂HPO₄, 2 mM NaN₃, 4.6 mM sodium trimethylsilyl propionate-[2,2,3,3-2H₄] (TSP) in 80% D₂O, pH 7.4±0.1). NMR SampleJet™ tubes were sealed with POM balls added to the caps and stored in 96 well plates. All processing procedures were compliant with previous recommendations on sample handling and storage for COVID-19 samples.

Quantification of plasma lipoproteins—A total of 112 lipoprotein parameters were quantified based on 1D NMR experiments as part of Bruker's IVDr experiment suite for blood plasma (Table 5). This approach is termed Bruker's IVDr lipoprotein class and subclass Analysis (B.I.-LISA™) and lipid analytes include cholesterol, free cholesterol, phospholipids, triglycerides, apolipoproteins A1/A2/B100 and ratio B100/A1, in total plasma concentration and resolved for main lipoprotein classes and subclasses. Main classes of plasma-lipoproteins were defined as: high-density lipoprotein (HDL, density 1.063-1.210 kg/L), intermediate-density lipoprotein (IDL, density 1.006-1.019 kg/L) low-density lipoprotein (LDL, density 1.09-1.63 kg/L) and very low-density lipoprotein (VLDL, 0.950-1.006 kg/L). The main lipoprotein classes HDL, LDL, VLDL were further divided into different lipoprotein sub-classes: (LDL-1:1019-1.031 kg/L, LDL-2: 1.031-1.034 kg/L, LDL-3: 1.034-1.037 kg/L, LDL-4: 1.037-1.040 kg/L, LDL-5: 1.040-1.044 kg/L, LDL-6: 1.044-1.063 kg/L), and the HDL sub-fractions into four density classes (HDL-1 1.063-1.100 kg/L, HDL-2 1.100-1.125 kg/L, HDL-3 1.125-1.175 kg/L, and HDL-4 1.175-1.210 kg/L), the VLDL sub-fractions divided into 5 density classes.

TABLE 5
Annotation of the keys used by the Bruker IVDr Lipoprotein
Subclass Analysis (B.I.-LISA ™) method.

| | | | Concentration |
|---|---|---|---|
| Key | Class/subclass | Compound | unit |
| TPTG | Total Plasma | Triglycerides | mg/dL |
| TPCH | Total Plasma | Cholesterol | mg/dL |
| LDCH | LDL | Cholesterol | mg/dL |
| HDCH | HDL | Cholesterol | mg/dL |
| TPA1 | Total Plasma | Apolipoprotein-A1 | mg/dL |
| TPA2 | Total Plasma | Apolipoprotein-A2 | mg/dL |
| TPAB | Total Plasma | Apolipoprotein-B100 | mg/dL |
| LDHD | Ratio LDL and HDL | LDL Cholesterol / | —/— |
| | Cholesterol | HDL Cholesterol | |
| ABA1 | Ratio of | Apolipoprotein-A1 / | —/— |
| | Apolipoproteins | Apolipoprotein-B100 | |
| | A1 and B100 | | |
| TBPN | Apolipoprotein-B100 | Particle Number | nmol/L |
| | carrying particles | | |
| VLPN | VLDL | Particle Number | nmol/L |
| IDPN | IDL | Particle Number | nmol/L |
| LDPN | LDL | Particle Number | nmol/L |
| L1PN | LDL-1 | Particle Number | nmol/L |
| L2PN | LDL-2 | Particle Number | nmol/L |
| L3PN | LDL-3 | Particle Number | nmol/L |
| L4PN | LDL-4 | Particle Number | nmol/L |
| L5PN | LDL-5 | Particle Number | nmol/L |
| L6PN | LDL-6 | Particle Number | nmol/L |
| VLTG | VLDL Class | Triglycerides | mg/dL |
| IDTG | IDL Class | Triglycerides | mg/dL |
| LDTG | LDL Class | Triglycerides | mg/dL |
| HDTG | HDL Class | Triglycerides | mg/dL |
| VLCH | VLDL Class | Cholesterol | mg/dL |
| IDCH | IDL Class | Cholesterol | mg/dL |
| LDCH | LDL Class | Cholesterol | mg/dL |
| HDCH | HDL Class | Cholesterol | mg/dL |
| VLFC | VLDL Class | Free Cholesterol | mg/dL |
| IDFC | IDL Class | Free Cholesterol | mg/dL |
| LDFC | LDL Class | Free Cholesterol | mg/dL |
| HDFC | HDL Class | Free Cholesterol | mg/dL |
| VLPL | VLDL Class | Phospholipids | mg/dL |
| IDPL | IDL Class | Phospholipids | mg/dL |
| LDPL | LDL Class | Phospholipids | mg/dL |
| HDPL | HDL Class | Phospholipids | mg/dL |
| HDA1 | HDL Class | Apolipoprotein-A1 | mg/dL |
| HDA2 | HDL Class | Apolipoprotein-A2 | mg/dL |
| VLAB | VLDL Class | Apolipoprotein-B100 | mg/dL |
| IDAB | IDL Class | Apolipoprotein-B100 | mg/dL |
| LDAB | LDL Class | Apolipoprotein-B100 | mg/dL |
| V1TG | VLDL-1 Subclass | Triglycerides | mg/dL |
| V2TG | VLDL-2 Subclass | Triglycerides | mg/dL |
| V3TG | VLDL-3 Subclass | Triglycerides | mg/dL |
| V4TG | VLDL-4 Subclass | Triglycerides | mg/dL |
| V5TG | VLDL-5 Subclass | Triglycerides | mg/dL |
| V1CH | VLDL-1 Subclass | Cholesterol | mg/dL |
| V2CH | VLDL-2 Subclass | Cholesterol | mg/dL |
| V3CH | VLDL-3 Subclass | Cholesterol | mg/dL |
| V4CH | VLDL-4 Subclass | Cholesterol | mg/dL |
| V5CH | VLDL-5 Subclass | Cholesterol | mg/dL |
| V1FC | VLDL-1 Subclass | Free Cholesterol | mg/dL |
| V2FC | VLDL-2 Subclass | Free Cholesterol | mg/dL |
| V3FC | VLDL-3 Subclass | Free Cholesterol | mg/dL |
| V4FC | VLDL-4 Subclass | Free Cholesterol | mg/dL |
| V5FC | VLDL-5 Subclass | Free Cholesterol | mg/dL |
| V1PL | VLDL-1 Subclass | Phospholipids | mg/dL |
| V2PL | VLDL-2 Subclass | Phospholipids | mg/dL |
| V3PL | VLDL-3 Subclass | Phospholipids | mg/dL |
| V4PL | VLDL-4 Subclass | Phospholipids | mg/dL |
| V5PL | VLDL-5 Subclass | Phospholipids | mg/dL |
| L1TG | LDL-1 Subclass | Triglycerides | mg/dL |
| L2TG | LDL-2 Subclass | Triglycerides | mg/dL |
| L3TG | LDL-3 Subclass | Triglycerides | mg/dL |
| L4TG | LDL-4 Subclass | Triglycerides | mg/dL |
| L5TG | LDL-5 Subclass | Triglycerides | mg/dL |
| L6TG | LDL-6 Subclass | Triglycerides | mg/dL |
| L1CH | LDL-1 Subclass | Cholesterol | mg/dL |
| L2CH | LDL-2 Subclass | Cholesterol | mg/dL |
| L3CH | LDL-3 Subclass | Cholesterol | mg/dL |
| L4CH | LDL-4 Subclass | Cholesterol | mg/dL |
| L5CH | LDL-5 Subclass | Cholesterol | mg/dL |
| L6CH | LDL-6 Subclass | Cholesterol | mg/dL |
| L1FC | LDL-1 Subclass | Free Cholesterol | mg/dL |
| L2FC | LDL-2 Subclass | Free Cholesterol | mg/dL |
| L3FC | LDL-3 Subclass | Free Cholesterol | mg/dL |
| L4FC | LDL-4 Subclass | Free Cholesterol | mg/dL |
| L5FC | LDL-5 Subclass | Free Cholesterol | mg/dL |
| L6FC | LDL-6 Subclass | Free Cholesterol | mg/dL |
| L1PL | LDL-1 Subclass | Phospholipids | mg/dL |
| L2PL | LDL-2 Subclass | Phospholipids | mg/dL |
| L3PL | LDL-3 Subclass | Phospholipids | mg/dL |
| L4PL | LDL-4 Subclass | Phospholipids | mg/dL |
| L5PL | LDL-5 Subclass | Phospholipids | mg/dL |
| L6PL | LDL-6 Subclass | Phospholipids | mg/dL |
| L1AB | LDL-1 Subclass | Apolipoprotein-B100 | mg/dL |
| L2AB | LDL-2 Subclass | Apolipoprotein-B100 | mg/dL |
| L3AB | LDL-3 Subclass | Apolipoprotein-B100 | mg/dL |
| L4AB | LDL-4 Subclass | Apolipoprotein-B100 | mg/dL |
| L5AB | LDL-5 Subclass | Apolipoprotein-B100 | mg/dL |
| L6AB | LDL-6 Subclass | Apolipoprotein-B100 | mg/dL |
| H1TG | HDL-1 Subclass | Triglycerides | mg/dL |
| H2TG | HDL-2 Subclass | Triglycerides | mg/dL |
| H3TG | HDL-3 Subclass | Triglycerides | mg/dL |
| H4TG | HDL-4 Subclass | Triglycerides | mg/dL |
| H1CH | HDL-1 Subclass | Cholesterol | mg/dL |
| H2CH | HDL-2 Subclass | Cholesterol | mg/dL |
| H3CH | HDL-3 Subclass | Cholesterol | mg/dL |
| H4CH | HDL-4 Subclass | Cholesterol | mg/dL |
| H1FC | HDL-1 Subclass | Free Cholesterol | mg/dL |
| H2FC | HDL-2 Subclass | Free Cholesterol | mg/dL |
| H3FC | HDL-3 Subclass | Free Cholesterol | mg/dL |
| H4FC | HDL-4 Subclass | Free Cholesterol | mg/dL |
| H1PL | HDL-1 Subclass | Phospholipids | mg/dL |
| H2PL | HDL-2 Subclass | Phospholipids | mg/dL |
| H3PL | HDL-3 Subclass | Phospholipids | mg/dL |
| H4PL | HDL-4 Subclass | Phospholipids | mg/dL |
| H1A1 | HDL-1 Subclass | Apolipoprotein-A1 | mg/dL |
| H2A1 | HDL-2 Subclass | Apolipoprotein-A1 | mg/dL |
| H3A1 | HDL-3 Subclass | Apolipoprotein-A1 | mg/dL |
| H4A1 | HDL-4 Subclass | Apolipoprotein-A1 | mg/dL |
| H1A2 | HDL-1 Subclass | Apolipoprotein-A2 | mg/dL |
| H2A2 | HDL-2 Subclass | Apolipoprotein-A2 | mg/dL |
| H3A2 | HDL-3 Subclass | Apolipoprotein-A2 | mg/dL |
| H4A2 | HDL-4 Subclass | Apolipoprotein-A2 | mg/dL |

Abbreviations: LDL—low-density lipoprotein; HDL—high-density lipoprotein; VLDL—very low-density lipoprotein; IDL—intermediate-density lipoprotein.

600 MHz Proton NMR Spectroscopy and In Vitro Diagnostic Experiments: NMR spectroscopic analyses were performed on a 600 MHz Bruker BioSpin Corp. Avance III HD spectrometer equipped with a 5 mm BBI probe and fitted with the Bruker SampleJet™ robot cooling system set to 5° C. A full quantitative calibration was completed prior to the analysis using a previously described protocol. A series of NMR experiments were performed, comprising Bruker's In Vitro Diagnostics research (IVDr) methods set, including: i) a standard 1D experiment with solvent pre-saturation; ii) a Carr-Purcell-Meiboom-Gill (CPMG) spin-echo experiment; and iii) a 2-Dimensional J-resolved experiment. The total experiment time was 12.5 minutes per sample. Data was processed in automation mode using Bruker Topspin™ 3.6.2 and ICON™ NMR to achieve phasing, baseline correction and calibration to TSP. Further regression experiments were performed to quantify 112 parameters of main plasma lipoprotein classes and subclasses (Bruker IVDr Lipoprotein Subclass Analysis, B.I.-LISA™) based on a PLS-regression model using the —(CH₂)ₙ (δ 1.25) and —CH₃ (δ 0.80) signal.

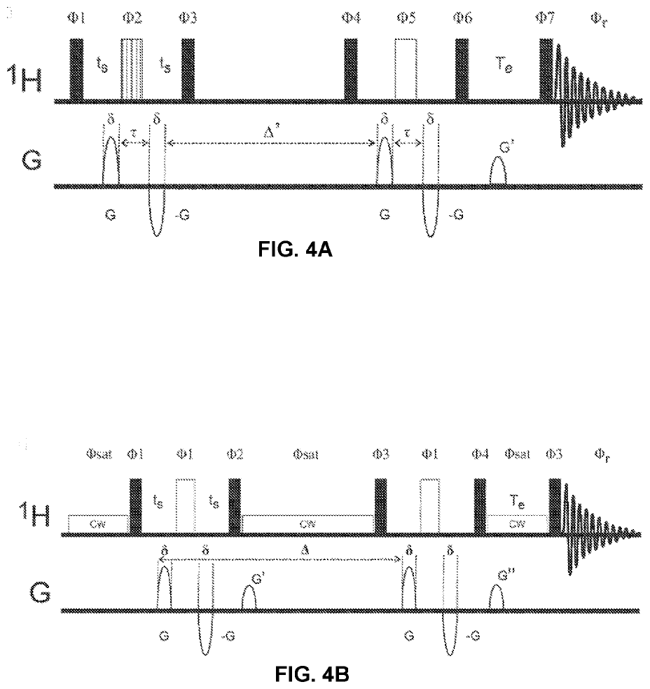

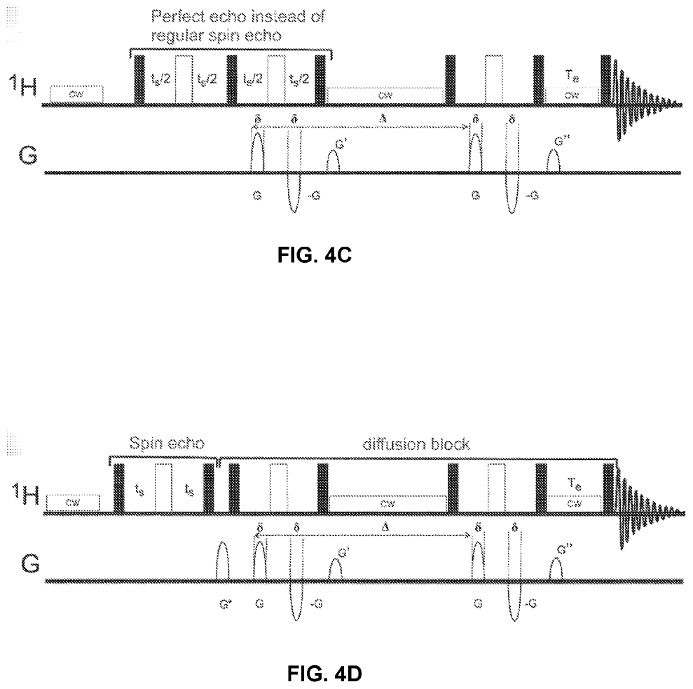

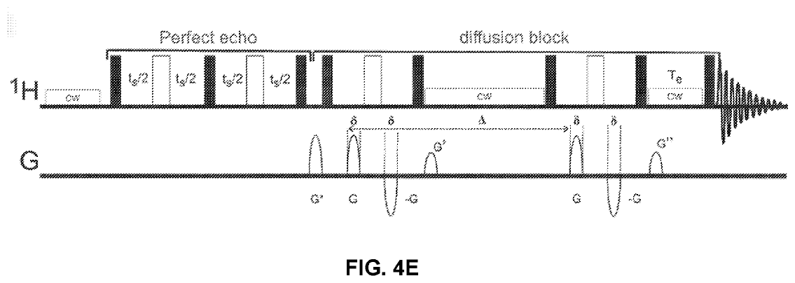

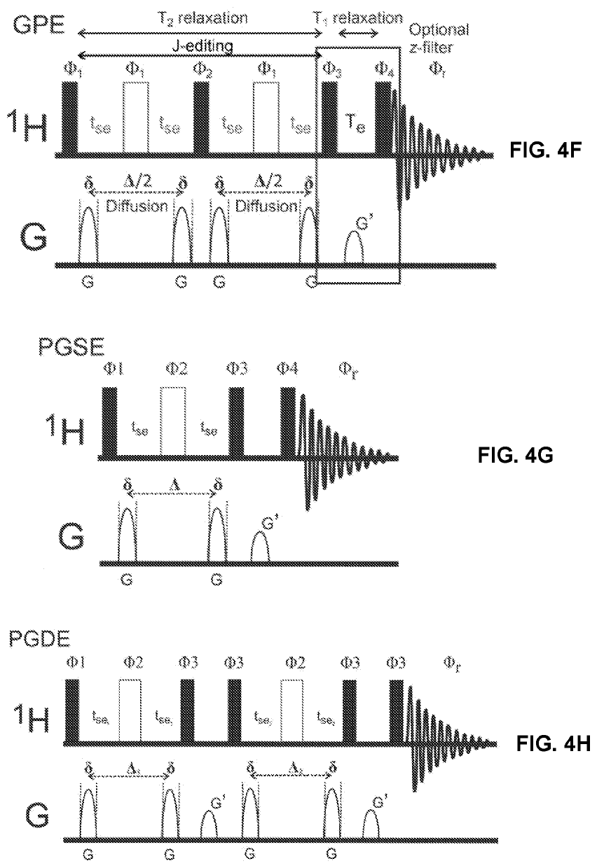

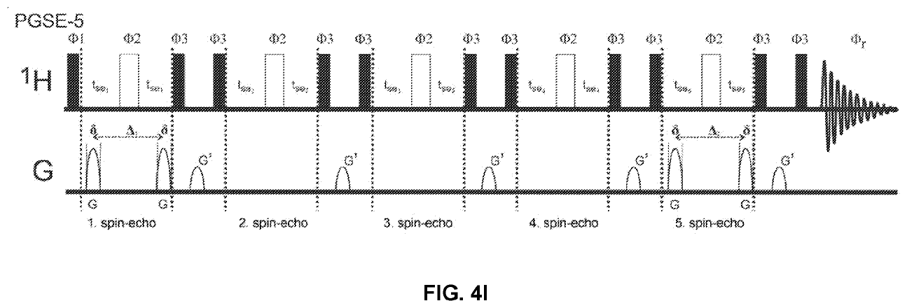

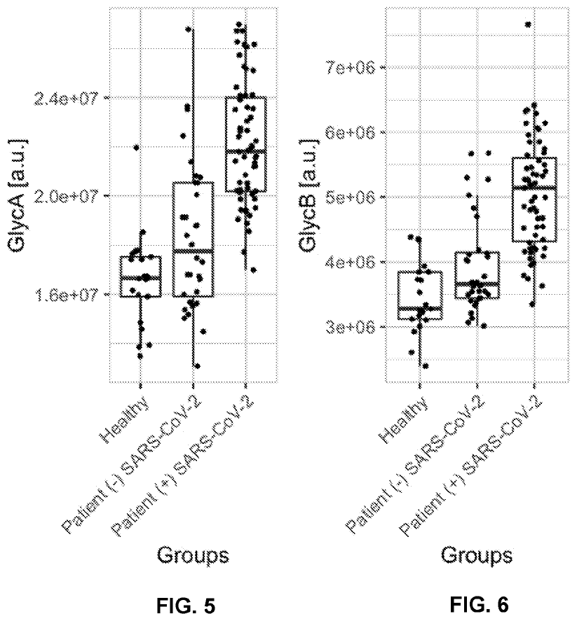

Diffusion and Relaxation Editing NMR Experiments: A Diffusion Relaxation Editing (DIRE) approach was applied to further investigate its diagnostic potential for assessment of SARS-CoV-2 positivity. FIGS. 4A-4I include both DIRE and JEDI sequences. Compared to a standard LED sequence, DIRE incorporates additional delays to allow for T₂ editing. A previously known DIRE sequence, referred to as DIRE-1, is shown in FIG. 4A. A series of additional DIRE pulse sequences were designed and tested, and are shown, respectively, in FIGS. 4B-4I. Each of these sequences has various irradiation and gradient modifications relative to the DIRE-1 sequence, and all gave broadly similar levels of spectroscopic classification and performance for detecting SARS-CoV-2 positivity.

The DIRE sequence used in the exemplary embodiment is shown in FIG. 4B. This modified DIRE pulse sequence was used because it is simple to implement for high throughput applications. For each sample, DIRE NMR experiments were completed in full automation with a total analysis time of 4.5 minutes (64 scans, 98K data points, spectral width of 30 ppm). All gave similar results and near-equivalent diagnostic classification. The DIRE variant employed in the study discussed herein replaced the previously-known modified WATERGATE solvent suppression sequence with a continuous secondary irradiation field at the water resonance frequency. For simplicity, this new sequence is referred to simply as DIRE throughout the subsequent text. To aid signal assignment, two-dimensional NMR methods and model phospholipid titrations into plasma were performed.

The other new sequences represent alternative examples that may be used with the invention. In the sequences shown in FIGS. 4C, 4D and 4E, the narrow bars are 90° pulses, the open rectangles are 180° pulses or composite 180° pulses (ϕ2 in FIG. 4A), and G′, G″ and G are smoothed-square shape gradients operating at −17.13%,-13.13% and 80% gradient strength, respectively, (maximum nominal gradient strength 53.5 G/cm). In addition, these sequences use an eddy current delay, Tₑ, of 5 ms, and 2tₛ is a 33 ms total spin-spin relaxation period. CW is on-resonance, continuous irradiation to saturate the water signal, and the diffusion delays δ and Δ were set to 1.5 ms and 120 ms, respectively. The phase cycling is ϕ1=x: ϕ2 =(x)₂, (−x)₂; ϕ3=(x)₄, (−x)₄, (y)₄, (−y)₄; ϕ4=x, −x, x, (−x)₂, x, −x, x, y, −y, y, (−y)₂, y, −y, y; ϕsat=x; ϕr=x, (−x)₂, x, −x, (x)2, −x, −y, (y)₂, −y, y, (−y)₂, y.

In FIG. 4C, instead of a regular spin echo, a perfect echo is used to address J-modulation effects. In FIG. 4D, the spin echo block acting as a T₂ filter and the LED diffusion block are separated. In FIG. 4E, a combination of the pulse sequences of FIGS. 4C and 4D introduce the perfect echo and separation of T₂ filter from the LED diffusion block. These three sequences yield comparable spectra, although the DIRE pulse sequence of FIG. 4E is less prone to signal modulation for long T₂ filters.

FIG. 4F shows a Pulsed Gradient Perfect Echo (PGPE) pulse sequence highlighting the three editing approaches from JEDI; relaxation, diffusion and J-editing. The narrow, black rectangular bars are non-selective 90° pulses and the open rectangles are 180° non-selective pulses. G and G′ are smoothed-square shape gradients, whereas G as the diffusion delay 5 is set to 2.5 ms with a strength of 80% and G′ is set to 600 ps with a strength of 70% (nominal gradient strength at 100%=53.5 g/cm). tse is a spin-spin relaxation period set to 27.5 ms. The total diffusion time A accumulates to 104.6 ms. Te is an optional eddy current delay of 5 ms. The phase cycling is ϕ1=x; ϕ2=y; ϕ3=x, −x; ϕ4=(x)2; ϕr=x, −x.

FIG. 4G shows a Pulsed Gradient Spin Echo (PGSE) pulse sequence. Narrow, black rectangular bars in the figure represent non-selective 90° pulses and the broad, white rectangular bar represents a non-selective 180° pulse. G and G′ are smoothed-square shape gradients, whereas G as the diffusion delay δ is set to 3.0 ms with a strength of 80% and G′ is set to 560 μs with a strength of 70% (nominal gradient strength at 100%=53.5 g/cm). tse is a spin-spin relaxation period set to 33 ms. The total diffusion time Δ accumulates to 63.2 ms. During the z-filter, an optional eddy current delay can be included with a duration of 5 ms. The phase cycling is ϕ1=x; ϕ2=y; ϕ3=x, −x, x, −x; ϕ4=(x)4; ϕr=x, −x, x, −x.

FIG. 4H shows a Pulsed Gradient Double Echo (PGDE) pulse sequence. Again, the narrow, black rectangular bars are non-selective 90° pulses and broad, white rectangular bars present non-selective 180° pulses. G and G′ are smoothed-square shape gradients, whereas G as the diffusion delay δ is set to 2.3 ms with a strength of 80% and G′ is set to 560 μs with a strength of 17.13% (nominal gradient strength at 100% =53.5 g/cm). tₛₑ₁ and tₛₑ₂ are spin-spin relaxation periods set to 37.5 and 60 ms respectively. The total diffusion time Δ accumulates to 190 ms. During the z-filter, an optional eddy current delay can be included with a duration of 5 ms. The phase cycling is ϕ1=(x)2, (−x)4, x(2); ϕ2=(y)2, (−y)4, (y)2; ϕ3=x; ϕr=(x)2, (−x)4, x(2).

FIG. 4I shows a Pulsed Gradient Spin Echo×5 (PGSE-5) pulse sequence. Narrow, black rectangular bars are non-selective 90° pulses and broad, white rectangular bars present non-selective 180° pulses. G and G′ are smoothed-square shape gradients, whereas G as the diffusion delay δ is set to 2.3 ms with a strength of 80% and G′ is set to 560 μs with a strength of 17.3% (nominal gradient strength at 100%=53.5 g/cm). tₛₑ₁-tₛₑ₅ are spin-spin relaxation periods set to 14.5, 20.5,37.5, 41.5, and 71.0 ms respectively. The total diffusion time A accumulates to 176 ms. During the z-filter in the 5th spin-echo an optional eddy current delay can be included with a duration of 5 ms. The phase cycling is ϕ1=(x)2, (−x)4, x(2); ϕ2=(y)2, (−y)4, (y)2; ϕ3=x; ϕr=(x)2, (−x)4, x(2).

Identification of Key Molecular Species in DIRE Spectral Signatures: DIRE spectra have a relatively small number of discrete and composite signal features consisting of a general triglyceride pattern with aliphatic side chains and typical signals from the methine (unsaturated) carbons, a strong N-acetyl signal attributed to acute phase reactive glycoproteins (mainly GlycA at δ 2.03), with a smaller but significant contribution from GlycB at δ 2.07, and a choline head group (⁺N—(CH₃)₃) signal at δ 3.20-3.30 from phospholipids including phosphatidyl-and lysophosphatidylcholines (also with aliphatic side chains overlapped with the triglycerides), that can be further decomposed into two major contributions from signals at δ 3.22 (SPC₁) and δ 3.26 (SPC₃); all of these signals are to a greater or lesser extent composite peaks as described below. To be expressed in a DIRE spectrum, the molecular species all share some commonality of molecular motion, diffusion and relaxation times.

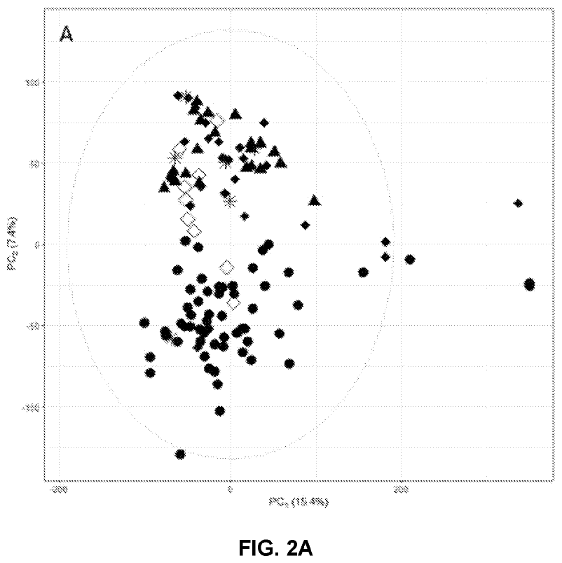

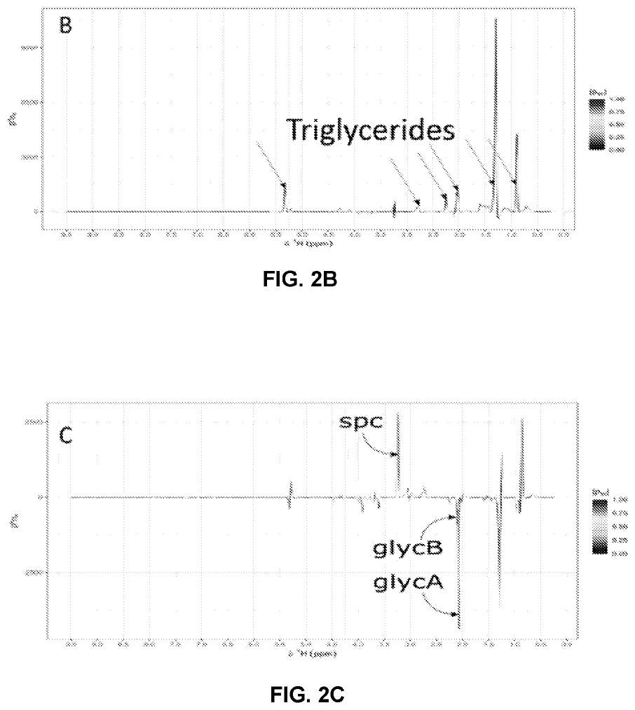

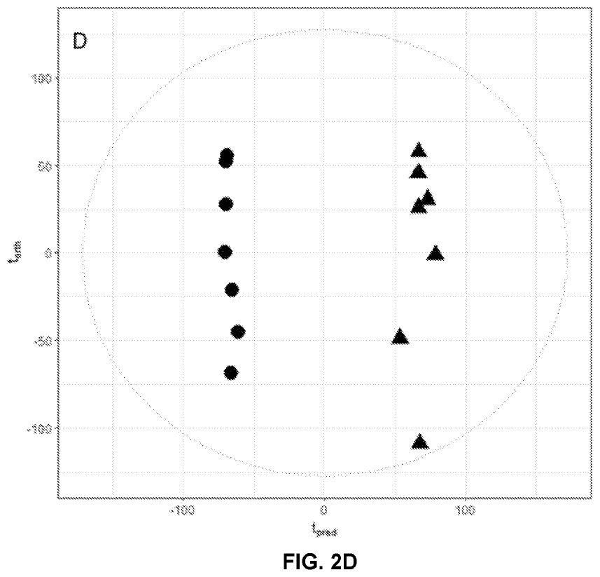

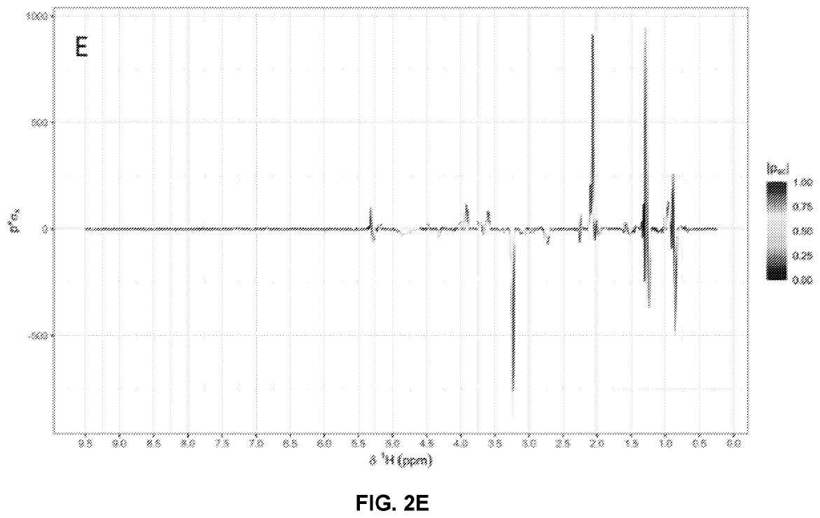

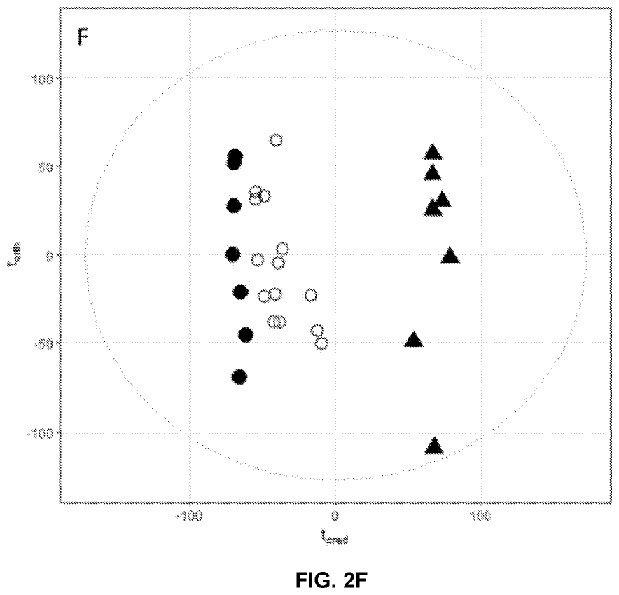

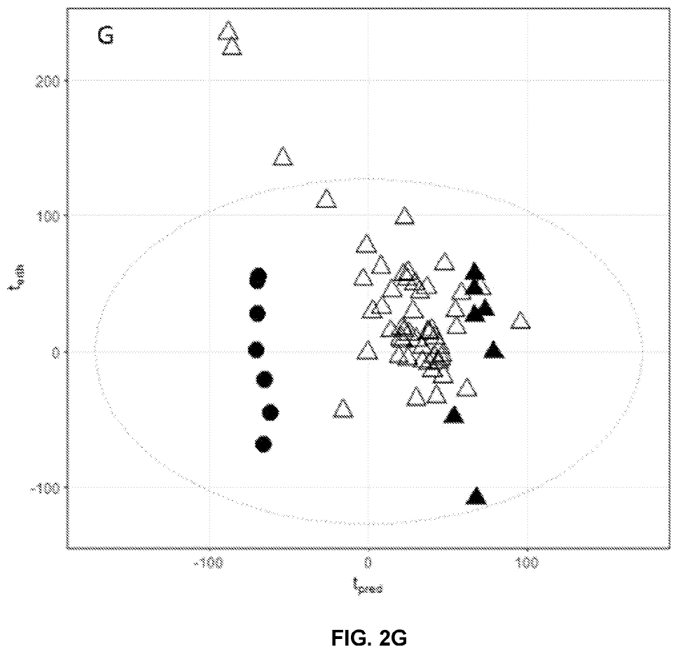

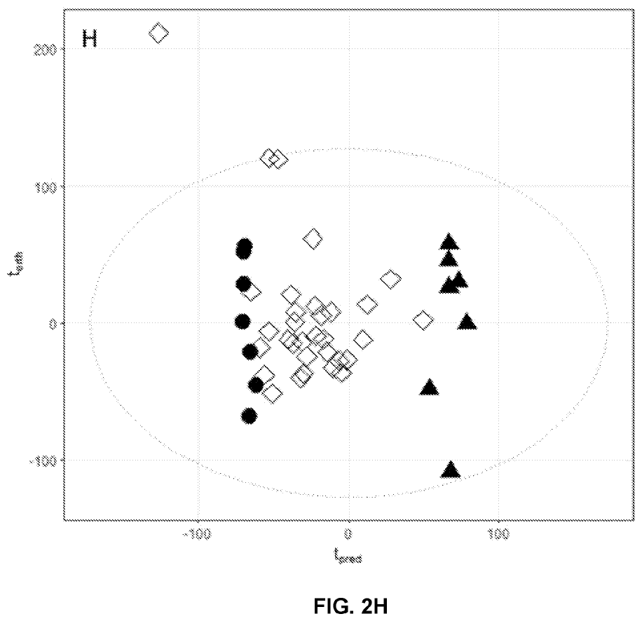

The GlycA signal contains contributions from α-1-acid glycoprotein, accounting for most of the intensity, with lesser contributions from a composite of signals from α-1-antichymotrypsin, α-1-antitrypsin, haptoglobin and transferrin. The role of α-1-acid glycoprotein in binding a range of lysophosphatidylcholines in a 1:1 molar ratio as well as small lipophilic molecules has previously been established. However, in contrast to GlycA and GlycB, which increase as a response to SARS-CoV-2 infection, most (lyso)-phosphatidylcholine species decrease. This contrasting behaviour is captured in the OPLS-DA coefficient plot (FIG. 2).

Data Pre-processing and Statistical Evaluation: 1D spectral data pre-processing comprised the excision of the residual water resonances (δ 4.5-5.0) and chemical shift regions with no signals of interest (δ<0.25 and δ>9.5). The chemical shift axis was calibrated to the a-anomeric proton of glucose (δ 5.23). A similar procedure was applied to CPMG spectra, whereas the chemical shift calibration of DIRE spectra reflects the frequency of the water suppression as no further calibration is performed. Other DIRE calibration options were tested using DIRE signals as calibrants. Spectra were baseline corrected using an asymmetric least squares method. To aid the assignment of structural information relating to DIRE signals δ 2.03 (GlycA) and 5 3.20-3.30 (SPC), 1D Statistical Total Correlation Spectroscopy (STOCSY) was applied using the DIRE spectra from all study groups (healthy controls, patients with and without SARS-CoV-2 infection). Statistical evaluation was performed with principal components analysis (PCA) as an unsupervised multivariate method, compressing the high-dimensional spectral data set into a few latent variables, thereby establishing potential sample clustering trends based on covariance structure. Group comparison was performed using orthogonal-projections to latent structures-discriminant analysis (O-PLS-DA) using a training set of seven SARS-CoV-2 positive and seven age and sex matched healthy controls. The optimal number of components (1 predictive+1 orthogonal) in the model was established using the area under the receiver operator characteristic curve (AUROC) as model generalisation index, computed in a jack-knifing statistical cross-validation framework. Data were mean-centred and scaled to unit-variance prior to multivariate modelling. All data analysis tasks were performed in the statistical programming language R, using the metabom8 package (V 0.2), obtainable at https://github.com/tkimhofer. In order to evaluate associations between GlycA, GlycB, SPC and plasma lipoproteins measured by IVDr, a Spearman's correlation analysis was performed using respective signal integrals. Results were visualized as heatmaps, rows and columns were ordered according to lipoprotein density classes. Features with low correlation coefficients are not shown.

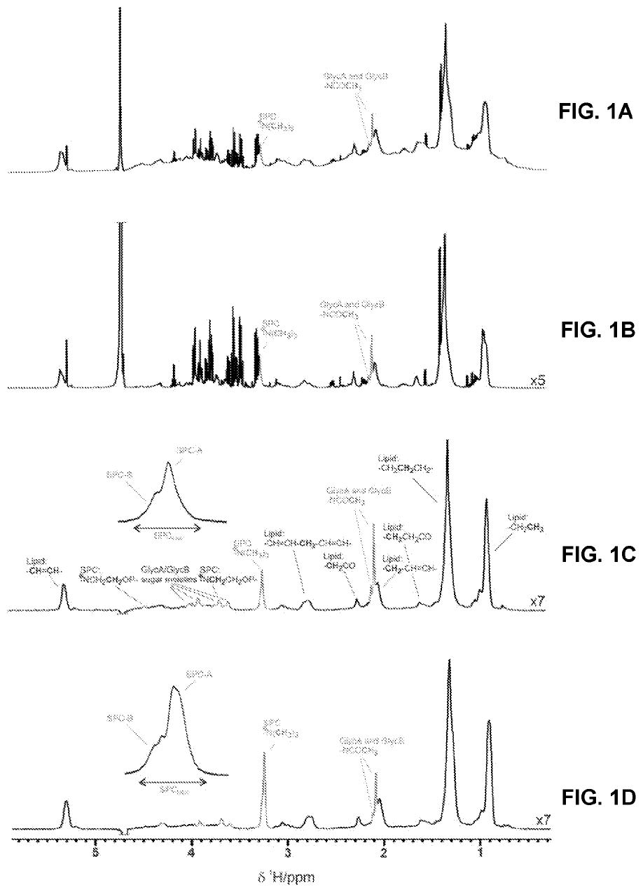

NMR Spectra of Blood Plasma: Typical water-suppressed 1 dimensional, spin-echo and DIRE spectra of plasma (control plus patient) are shown in FIGS. 1A-1D. The DIRE ¹H NMR spectra provide an observational window on molecules and structures with slow translational diffusion, but high levels of segmental motion (long T₂ relaxation times) capturing the compositional differences in certain plasma glycoproteins and lipoprotein sub-compartments (FIGS. 1C and 1D). Thus, in comparison to standard 1-D 600 MHz ¹H NMR water-suppressed spectra (FIG. 1A), which are dominated by broad envelopes of macromolecular resonances with superimposed sharp signals mainly from low molecular weight metabolites, it is easier to see the contributions from N-acetyl glycoproteins and selected compartmentalized lipoprotein signals.

The lipid peaks in FIGS. 1C and 1D are labelled according to chemical structure rather than from their lipoprotein components. In these DIRE spectra, the composite lipidic signals are dominantly from phosphatidylcholine and other phospholipids in HDL, bound to glycoprotein and, to a lesser extent, LDL. The main DIRE COVID-19 discriminating peaks are identified and represent N-acetylglycoprotein signals GlycA and GlycB and SPC, which refers to a supramolecular phospholipid composite —N⁺(CH₃)₃. Signal SPCₜₒₜₐₗ refers to the integrated signal from the choline head groups in the SPC. FIGS. 1C and 1D show a significant difference in peak intensities of GlycA and GlycB of the healthy cohort (FIG. 1D) compared to the SARS-Cov positive group FIG. 1C), i.e. the intensities of the Glyc peaks are higher in in the samples of the SARS-Cov positive cohort.

With respect to molecular information, the diffusion edited spectra, in which signals from molecules with rapid translational diffusion are eliminated, are almost the reverse of the CPMG spin echo spectra (FIG. 1B) that only carry information on sharp line signals from protons with long T₂ relaxation times. Whilst standard 1D and CPMG pulse sequences have previously been applied to blood plasma in multiple physiological and pathological studies to extract molecular biomarker information, diffusion-edited spectroscopy has been less frequently applied in a diagnostic setting and DIRE spectroscopy has only been demonstrated in principle as a spectral editing tool, but not so far applied to study disease diagnostics or systemic pathology. Here, the normal healthy and SARS-CoV-2 positive samples were significantly different based on their DIRE NMR signatures, with increased intensity of the GlycA and GlycB signals and decreased intensity of a cluster of choline head group signals at δ 3.22.

Multivariate Statistical Analysis of DIRE Spectra: FIG. 2A shows a Principal Components Analysis (PCA) of the DIRE spectra for SARS-CoV-2 PCR-positives, healthy and SARS-CoV-2 PCR-negatives together with spectra from sero-positive individuals who did not undergo PCR testing for SARS-CoV-2 infection. The figure uses triangles to represent healthy controls, circles to represent SARS-CoV-2 positive patients, black diamonds to represent SARS-CoV-2 negative non-hospitalised patients, white diamonds to represent SARS-CoV-2 negative hospitalised patients, and stars to represent serology (+) participants. The elliptical region indicates Hotelling's T₂ statistic (α=0.95), which can be interpreted as the multivariate confidence interval. As shown, there is a non-overlapping distribution for SARS-CoV-2 positives vs healthy, with individuals presenting with respiratory symptoms but PCR negative mapping closer to controls. Individuals that had tested seropositive but had not been tested for COVID-19 disease mapped within the space occupied by the control group, with the exception of one individual who mapped with the SARS-CoV-2 positive cluster. This individual had suffered a bout of extreme fatigue after an international trip four months earlier and was post hoc diagnosed as having COVID-19 with metabolic abnormalities at the time of testing.

Whereas the sample distribution in the first principal component (PC) was attributable to variation in triglycerides, as shown in the plot of the loadings of principal component 1 in FIG. 2B, the inherent differences in the molecular composition of plasma from SARS-CoV-2 PCR-positive patients and controls was defined in the second PC and the vector relating to SARS-CoV-2 infection was driven by increased intensity of the N-acetyl glycoprotein signals GlycA and GlycB and decreased intensity of a signal representing a phospholipid composite ⁺N—(CH₃)₃, as shown in the plot of principal loadings of principal component 2 in FIG. 2C. In order to clarify further the key molecular drivers of the differential SARS-CoV-2 signature, the data were modelled using OPLS-DA (1 predictive+1 orthogonal component) using a relatively small subset of the control (n=7) versus SARS-CoV-2 positive (N=7) spectral dataset. This is shown in FIG. 2D, while FIG. 2E shows OPLS-DA loadings of the training model.

Strong differentiation between the two groups and the projection of the remaining healthy (n=21) and SARS-CoV-2 positive (n=51) patients onto the training set (FIGS. 2F and 2G respectively) effectively achieved perfect classification (AUROC=1). The projections of the COVID-19 negative and the “recovered” seropositive individuals are shown in FIG. 2H and 2I and do not classify well with either healthy or SARS-CoV-2 positive training set clusters as would be expected, indicating that they were biochemically distinct from both the healthy controls and SARS-CoV-2 positive infected groups. The OPLS-DA model confirmed the observation from the PCA model that the main signature for SARS-CoV-2 positivity was dominated by signals relating to three main entities GlycA, GlycB (both higher in SARS-CoV-2 positive individuals) and the composite phospholipid signal, SPC (higher in healthy controls). NMR and statistical correlation tools were employed to further elucidate the molecular components of the SPC peaks and establish the statistical relationships between the candidate biomarker peaks.

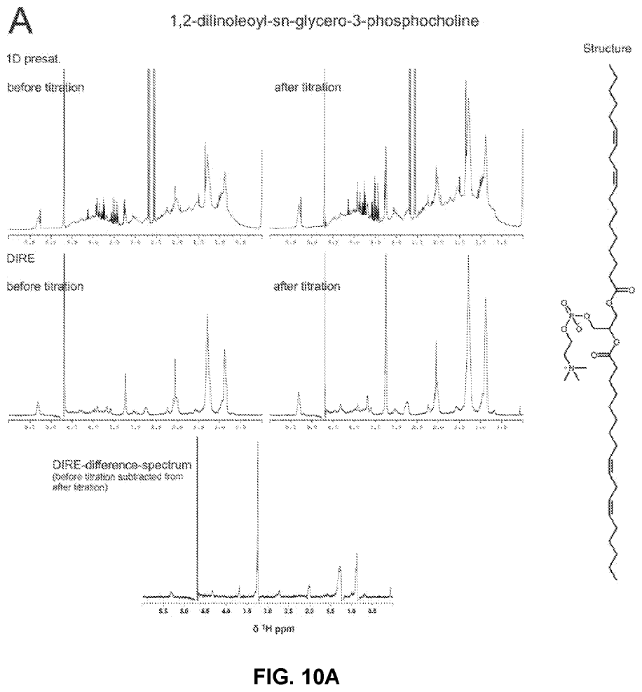

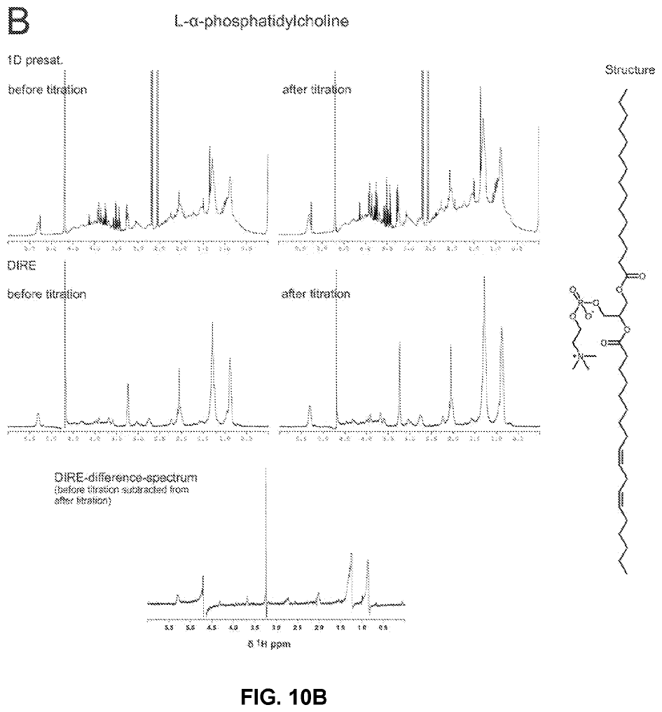

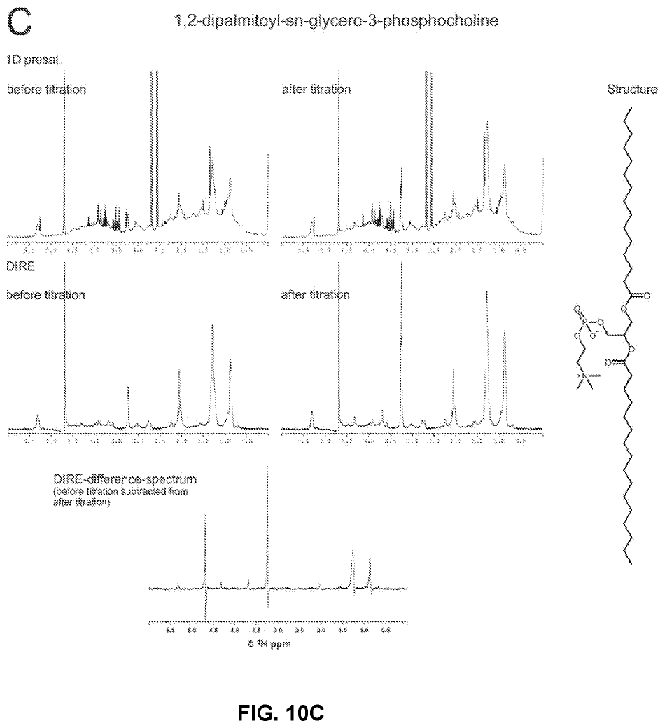

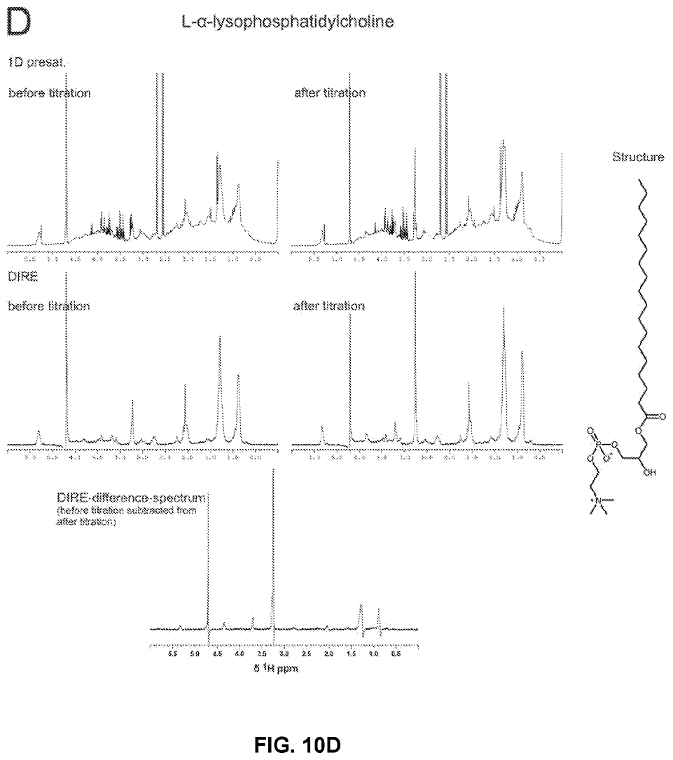

Differential Diagnostic Information in DIRE Spectra: DIRE spectra of blood plasma give a clear and unequivocal modelling diagnostic for SARS-CoV-2 infection. Thus, the key molecular contributors to the SARS-CoV-2 diagnostic in the diffusion-edited and ordered spectra were the N-acetyl glycoprotein peaks GlycA and GlycB and one of the major components of the composite DIRE signals at δ 3.22. This signal comes partly from a molecule with the same molecular diffusion constant as GlycA and GlycB, the most likely being linoleoylphosphatidylcholines based on its chemical shifts and known abundance (as shown in FIGS. 10A-10D, 11 and 12, discussed in more detail below). A second component of the composite signal δ 3.26 is likely to be other phospholipids present in high density lipoprotein domains based on the statistical correlations with the IVDr-derived lipoprotein data from the same samples.

The DIRE spectra show strong signals from these phosphatidylcholine species including the N⁺—(CH₃)₃ head groups of compartmentalized phospholipids and signals from partially unsaturated fatty acid side chains. As the summed N⁺—(CH₃)₃ signals in DIRE spectra have multicomponent origins (analogous to the GlycA and GlycB N-acetyl singlets), they are referred to as the Supramolecular Phospholipid Composite signals (SPC). The summed SPC integrals (SPCₜₒₜₐₗ) were used in statistical analysis of the relationships with the GlycA and GlycB signals in control and SARS-CoV-2 negative and SARS-CoV-2 positive patients are shown below in Table 6.

TABLE 6

| | | | SARS-CoV-2 | P value | P value | P value |
|---|---|---|---|---|---|---|
| | | SARS-CoV-2 | positive patients | Controls vs | Controls vs | SARs-CoV-2 |
| Spectral | Healthy Controls | negative patients | (n = 17, | SARS-CoV-2 | SARS-CoV-2 | negative vs |
| variable | (n = 26) | (n = 34) | 58 samples) | negative | positive | positive |
| GlycA | 1.67 × 10⁷ | 1.78 × 10⁷ | 2.18 × 10⁷ | 0.11 (NS) | 2.52 × 10⁻¹⁰ | 4.94 × 10⁻⁶ |
| (intensity) | [1.35 × 10⁷-2.20 × 10⁷] | [1.31 × 10⁷-2.68 × 10⁷] | [1.70 × 10⁷-2.70 × 10⁷] | | | |
| GlycB | 3.28 × 10⁶ | 3.66 × 10⁷ | 5.15 × 10⁶ | 0.14 (NS) | 1.25 × 10⁻⁹ | 9.24 × 10⁻⁹ |
| (intensity) | [2.40 × 10⁶-4.39 × 10⁶] | [3.01 × 10⁶-5.68 × 10⁶] | [3.35 × 10⁶-7.66 × 10⁶] | | | |
| GlycA plus | 2.00 × 10⁷ | 2.18 × 10⁷ | 2.63 × 10⁷ | 0.09 (NS) | 1.64 × 10⁻¹⁰ | 4.86 × 10⁻⁷ |
| GlycB | [1.64 × 10⁷-3.44 × 10⁷] | [1.62 × 10⁷-3.02 × 10⁷] | [2.13 × 10⁷-3.44 × 10⁷] | | | |
| (intensity) | | | | | | |
| SPCₜₒₜₐₗ | 3.72 × 10⁷ | 3.53 × 10⁷ | 2.62 × 10⁷ | 0.60 (NS) | 1.40 × 10⁻⁷ | 4.52 × 10⁻⁸ |
| (intensity) | [2.52 × 10⁷-4.61 × 10⁷] | [2.38 × 10⁷-5.12 × 10⁷] | [1.54 × 10⁷-4.15 × 10⁷] | | | |
| SPCₜₒₜₐₗ: | 2.05 | 1.98 | 1.19 | 0.21 (NS) | 1.23 × 10⁻¹⁰ | 1.60 × 10⁻⁹ |
| GlycA | [1.61-3.03] | [1.06-3.30] | [0.67-1.91] | | | |

Table 6 shows relative intensity group medians for GlycA, GlycB, SPC signal variables and their ratios, and Kruskal-Wallis rank sum test p values for differences between healthy controls and SARS-CoV-2 positive patients and SARS-CoV-2 negative patients vs SARS-CoV-2 positive patients. All p values shown in the table were determined using the Kruskal-Wallis rank sum test. GlycA and GlycB refer, respectively, to N-acetyl glycoprotein fragments A and B, and SPCₜₒₜₐₗ refers to the supramolecular phospholipid complex total signal. The label NS indicates “not significant.”

The triglyceride signals observed in DIRE spectra carried little direct diagnostic information being more closely reflective of body mass index than infection status. On closer inspection of the NMR data, SPC was seen to be composed of two major signals with separate average chemical shifts and linewidths (as shown in FIG. 1). These peaks are designated as SPC-A and SPC-B in an analogous fashion to the composite peak designation of GlycA and GlycB, and it was deduced that these originated from the different contributions from the LDL, HDL/glycoprotein bound phospholipids in each signal. Whilst the glycoprotein components show a strong positive association with SARS-CoV-2 infection, the total terminal head group phosphatidylcholine signals SPCₜₒₜₐₗ at δ 3.20-3.30 are significantly reduced in SARS-CoV-2 positive patients in comparison with controls (FIG. 1). α-1-acid glycoprotein and related acute phase reactive proteins are elevated in a variety of inflammatory conditions, and significant elevations in SARS-CoV-2 positive patients have been reported.

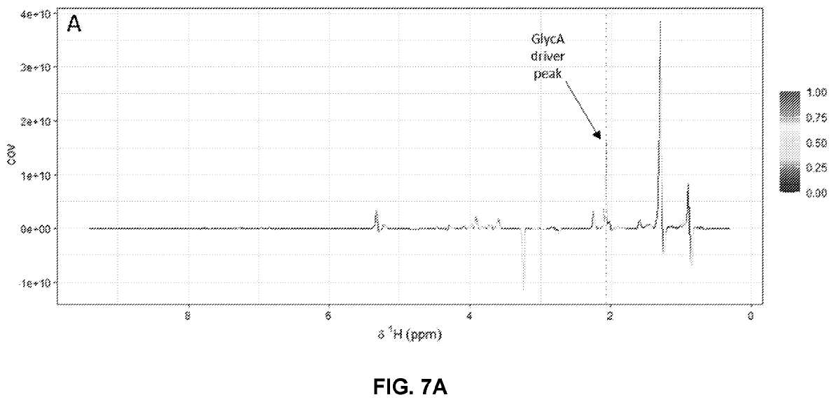

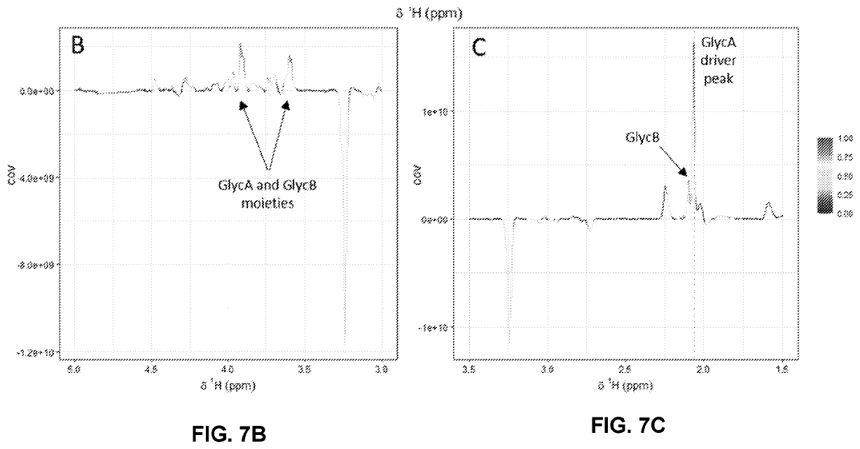

Statistical Total Correlation Spectroscopic Analysis of DIRE Spectra: Statistical TOtal Correlation SpectroscopY (STOCSY) allows structural connectivity to be established based on the covariance of proton signals from the same molecules across a series of spectra collected in parallel. STOCSY analysis of the DIRE spectra using the GlycA signal (δ 2.03) as the statistical driver peak statistically illuminating various other structurally correlated signals from other glycan protons from GlycA that can be observed in the region from δ 3.5 to 4.3 with the highest correlations (>0.9) at δ 3.7 and δ 3.9 are shown in FIGS. 7A-C and belong to various sugar ring moieties from the oligosaccharide chains of GlycA and related glycoproteins. FIG. 7A depicts the 1D STOCSY of the DIRE spectra showing the correlations for each spectral feature with respect to the selected peak (driver peak) at δ 2.03 (GlycA). FIG. 7B shows an expanded region δ 3.0-5.0, indicating strong correlation of the driver peak (GlycA) with peaks at δ 3.69 and δ 4.34. FIG. 7C shows an expanded region δ 1.5-3.5, indicating strong correlation of the driver peak (GlycA) with GlycB (δ 2.07). All analysis was completed with all samples in the spectral dataset, including healthy controls, SARS-CoV-2 positive patients and SARS-CoV-2 negative patients. GlycB can be observed highly correlated at δ 2.07, which is expected as GlycB presents the different acetyl residues (here: N-acetylneuraminidino groups) located on the same proteins that also generate the GlycA signal.

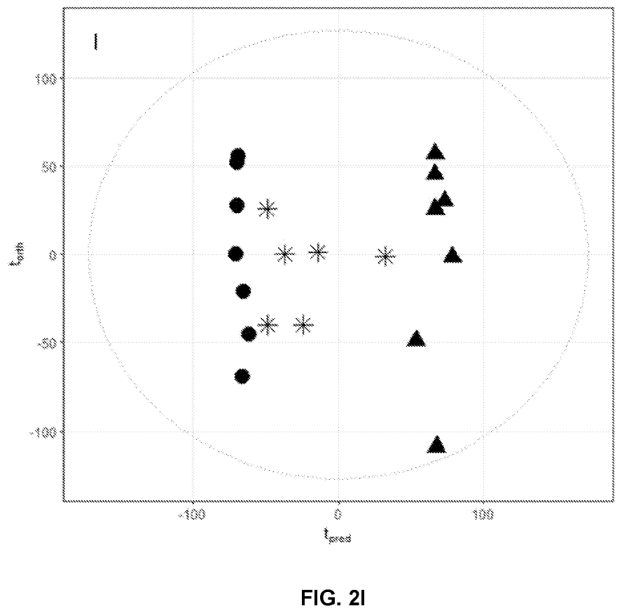

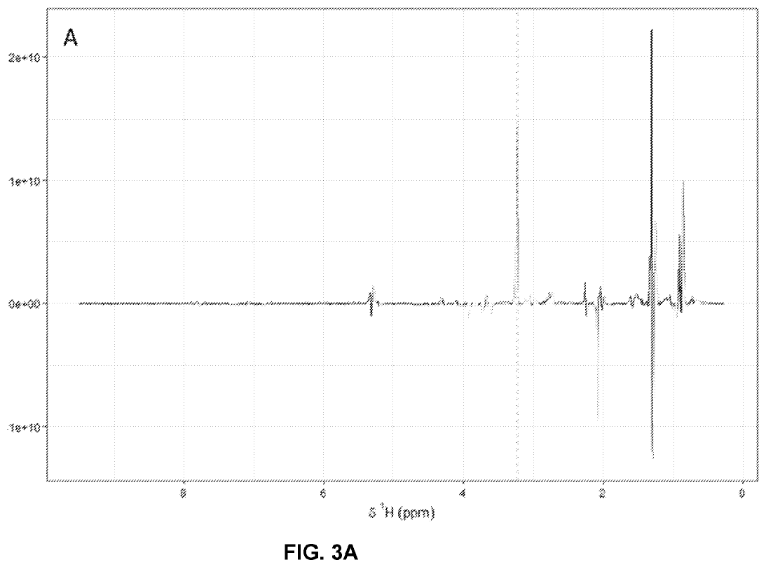

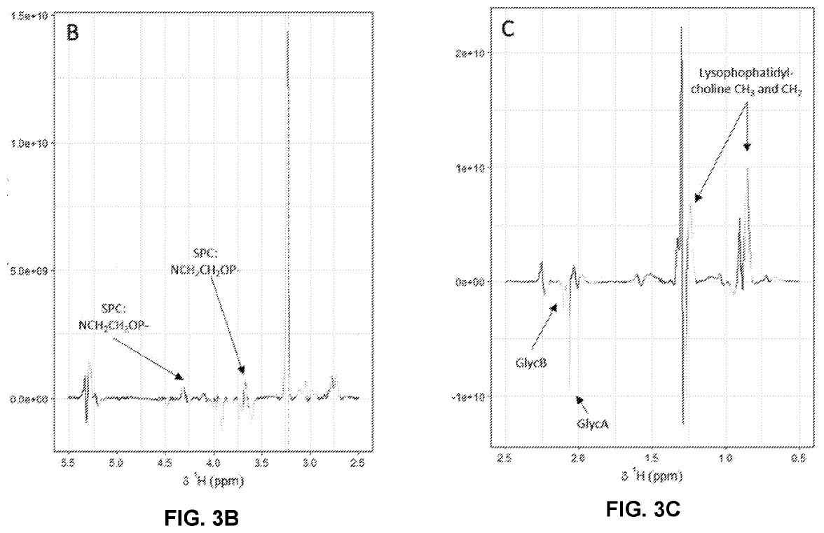

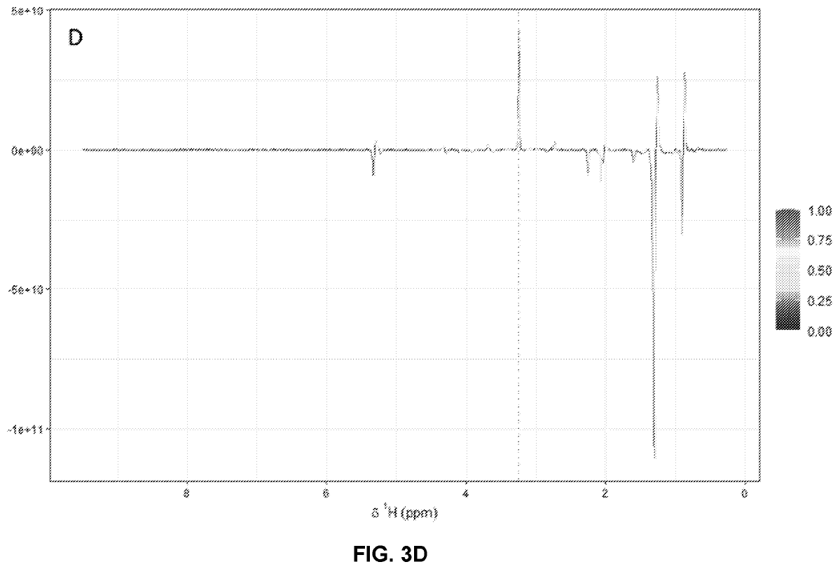

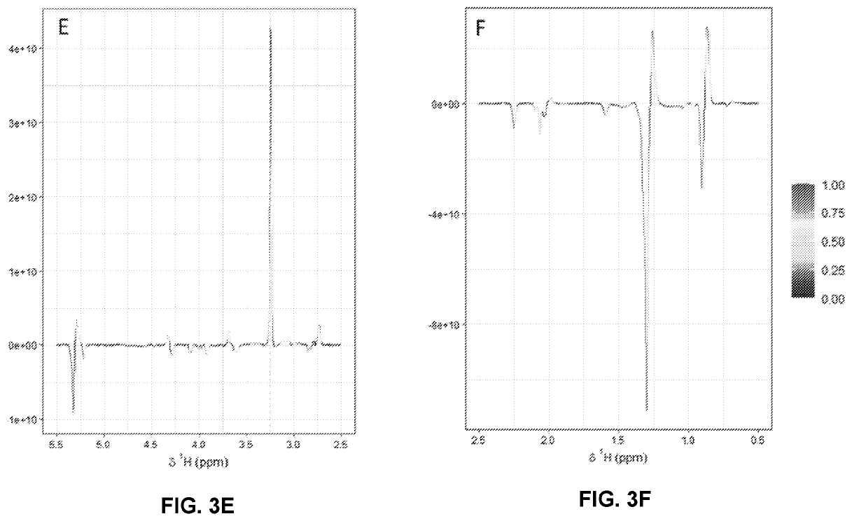

As shown in FIGS. 3A-3F, STOCSY analysis driven from the SPC-A signal at δ 3.22 shows a more extensive correlation landscape, including a highly correlated group of signals at δ 0.80, 1.25, 1.90, 2.7, 3.71, 4.0 and 5.4. Broad resonances around δ 3.2 in 1D plasma proton NMR spectra present trimethylammonium headgroups of choline moieties from phospholipids, e.g., Phosphatidylcholines or Sphingomyelins. FIG. 3A shows a 1D STOCSY of the full DIRE spectra using the SPC-A peak as the driver peak to determine the peaks highly correlated (gray). FIG. 3B shows an expanded region δ 2.5-5.5, indicating strong correlation of the driver peak (SPC-A peak) with peaks at δ 3.69 and 4.34. FIG. 3C shows an expanded region δ 0.5-2.5, indicating strong correlation of the driver peak (SPC-A peak δ 3.2-3,3) with two other peaks at δ 0.8 and δ 1.2. FIG. 3D shows a 1D STOCSY of the full DIRE spectra using the SPC-B peak as the driver peak to determine the peaks highly correlated (gray). FIG. 3E shows an expanded region δ 2.5-5.5,indicating strong correlation of the driver peak with peaks at δ 3.69 and 4.34. FIG. 3F shows an expanded region δ 0.5-2.5, indicating strong correlation of the driver peak, SPC-B with peaks at δ 0.8 and δ 1.2.

The observed STOCSY signal pattern is in good agreement with the expected chemical shifts for phosphatidylcholines as the signals at δ 3.69 and δ 4.34 correspond to the methylene groups in the choline moiety and the remaining signals belong to the attached alkyl chain. Notably, the shift for the signals at δ 0.8 and δ 1.3 matches the reported shifts for HDL particles, indicating that the STOCSY-highlighted phosphatidylcholine might be incorporated in HDL. Special attention is given to the complex multiplet signal at ca. δ 2.7 (FIG. 3B) as it is characteristic for a methylene (sp³ carbon) group in between the two sp² methine carbons, indicating that at least one linoleoyl (lyso-)phosphatidylcholine species is observed in these STOCSY data. This is consistent with known binding propensities of α₁-acid-glycoprotein and the observation of such bound species.

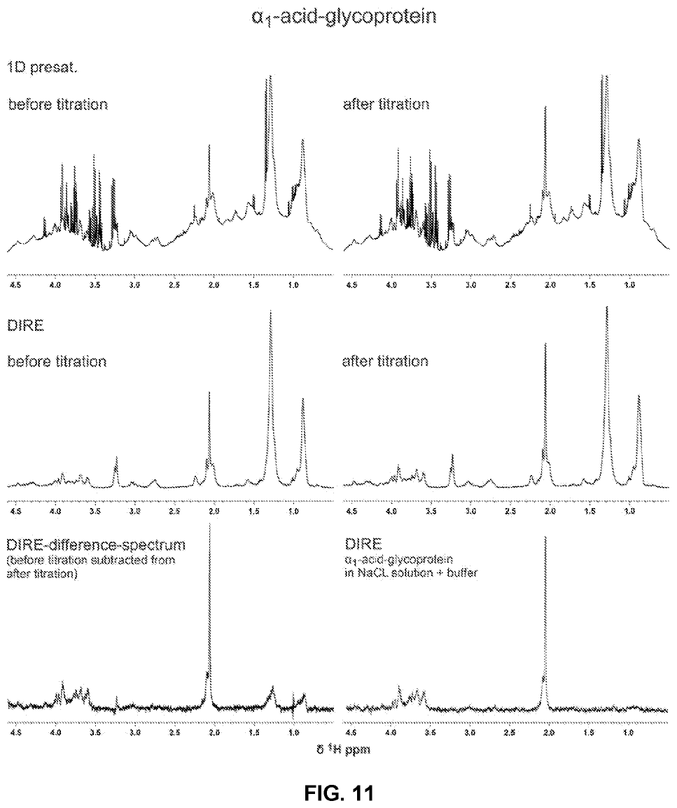

To corroborate the results from the STOCSY analysis, titrations of potential candidate molecules matching the observed signals were carried out. FIG. 11 depicts an NMR plot of ¹H 1D presat. and DIRE (top two spectra) with overlapping traces indicating the signal before titration (left) and after titration (right) of α₁-acid glycoprotein standard solution (16 μL of 9.6 mg al-acid glycoprotein in 70 μL NaCl solution). The resulting DIRE difference spectrum is also depicted, and shows the NMR signature of α₁-acid glycoprotein in plasma. The titration of α₁-acid glycoprotein results in a signal increase at δ 2.03 (Glu. Cal.) and δ 2.07 (Glu. Cal.) corresponding to the composite signals of GlycA and GlycB as the α₁-acid glycoprotein presents its main composite as described previously. Additionally, a set of signals between δ 3.5 and 4.1 can be observed, corresponding to various sugar moieties of the oligosaccharides from the glycoprotein. Furthermore, the DIRE difference spectrum shows a small but distinct resonance at δ 3.2 corresponding to the upfield part of the SPC signal and two broad resonances at δ 0.9 and δ 1.3 further suggesting an interaction of the α₁-acid glycoprotein with phosphatidylcholines and HDL particles. As mentioned in [0015], all the chemical shifts are reported after calibration to Glucose at δ 5.23 ppm.

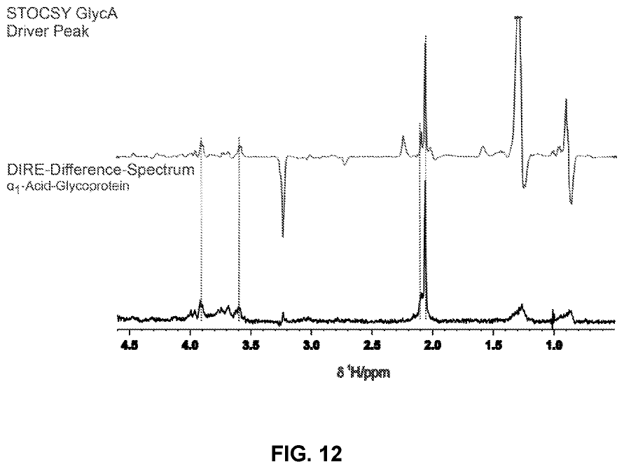

FIG. 12 shows a comparison of STOCSY with GlycA (δ 2.03) as the driver (upper spectrum) and the DIRE-difference spectrum with α₁-Acid-Glycoprotein as standard, and indicates a good overlap of both spectra confirming that some signals in a DIRE spectrum come from the various glycoproteins that comprise GlycA and GlycB. Furthermore, the DIRE difference spectrum shows a small but distinct resonance at δ 3.22 corresponding to SPC-A and two broad resonances at δ 0.9 and δ 1.3, further suggesting a possible binding interaction of the α-₁-acid glycoprotein with phosphatidylcholines and HDL particles.

In order to confirm the identity of the choline moiety unambiguously, a set of hetero-and homonuclear NMR experiments were performed: ¹H-¹H Diffusion Edited Total Correlation Spectroscopy (DE-TOCSY); ¹H-¹³C Heteronuclear Single Quantum Coherence (HSQC); and ¹H-¹³C Heteronuclear Multiple Bond Correlation (HMBC)). For DE-TOCSY, pre-processed plasma samples were analysed at 310 K. Bruker BioSpin Corp. pulse program ledbpgpm12s2dp, with an additional pre-saturation in sequence with 8 scans, 32 dummy scans, 512 data points in the F1 dimension, 2048 data points in the F2 dimension. D1=1.5 s, spectral width 13 ppm, O1 at 4.7 ppm, D9=90 ms, big Delta=150 ms, little Delta=3 ms z-field gradient strength 26.75 G/cm. HSQC samples prepared as described herein were analysed at 310 K. Pulse program: hsqcedetgpisp2.3 with 76 scans, 32 dummy scans, 512 data points in the F1 dimension, 4096 data points in the F2 dimension. D1=2 s, spectral width in the F1 dimension was 190 ppm, spectral width in the F2 dimension was 16 ppm. O1 in the F1 dimension was 90 ppm and O1 in the F2 dimension was 4.7 ppm. Total experiment time was 24 hours and 18 minutes. HMBC samples were prepared as described herein, and analysed at 310 K. Pulse program: hmbcetgpl3nd with additional presaturation in sequence with, 72 scans, 16 dummy scans, 512 data points in the F1 dimension, 4096 data points in the F2 dimension. D1=2 s, spectral width in the F1 dimension was 230 ppm, spectral width in the F2 dimension was 13 ppm. O1 in the F1 dimension was 105 ppm and O1 in the F2 dimension was 4.7 ppm. Total experiment time was 24 hours and 13 minutes.

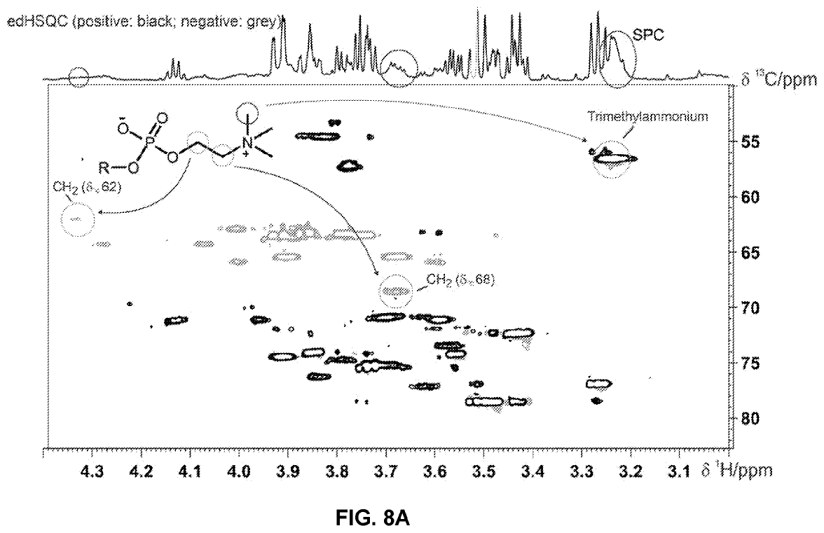

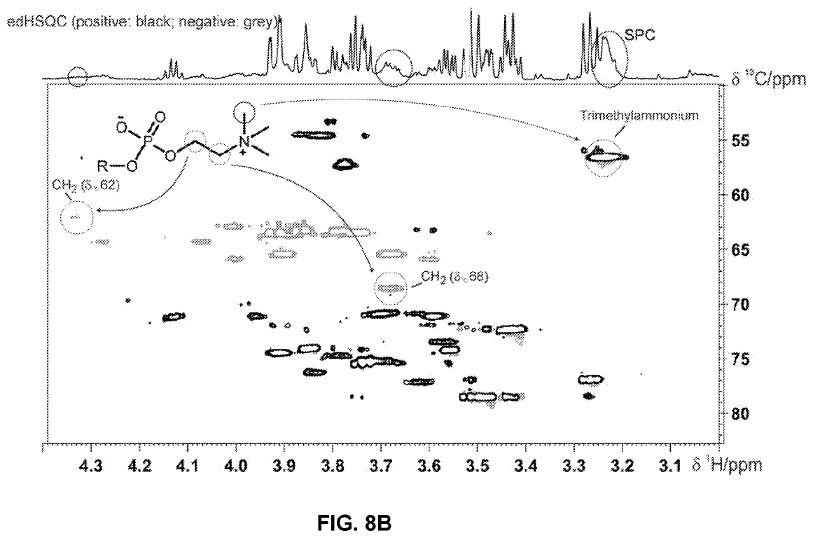

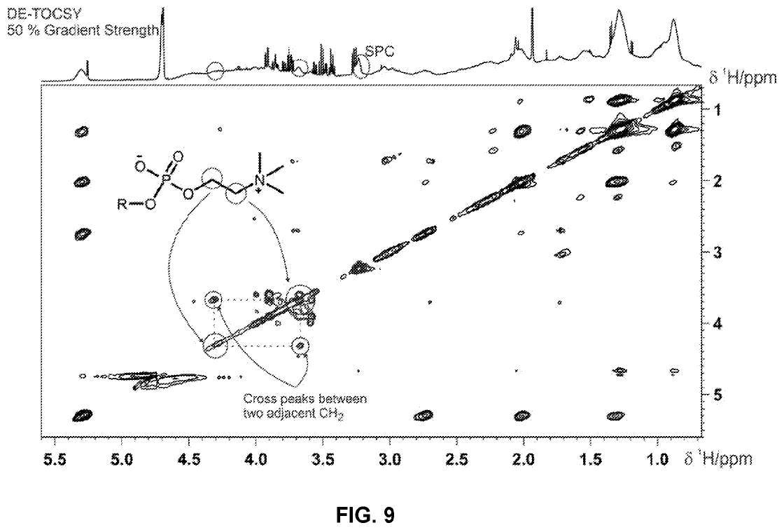

First, the ¹H¹³C edited HSQC NMR plot of FIG. 8A and the ¹H¹³C-HMBC NMR plot of FIG. 8B show a clear HMBC cross-peak to δ ¹³C 68.6 between the choline headgroup —N⁺—(CH₃)₃ signal belonging to SPC at δ ¹³C 56.6 and the closest methylene group in the choline moiety. In these figures, positive signals CH and CH₃ are shown in black, while negative signals CH₂ are shown in gray. The characteristic trimethylammonium signal, represented as a gray circle at 55 ppm in the HSQC plot, shows a clear cross peak to 68 ppm, represented as a gray circle in the HMBC plot, belonging to the nearest methylene group in the choline moiety (a gray circle at 68 ppm in the HSQC plot). The second methylene group of the choline moiety can be found at 62 ppm, shown as a gray circle at 62 ppm in the HSQC plot. All chemical shifts agree with literature shifts for phosphocholines. Secondly, a Diffusion Edited Total Correlation (DE-TOCSY) NMR spectrum (at 50% gradient strength, where 100%=53.5 G/cm), depicted in FIG. 9, was acquired that shows, at higher gradient strength, a correlation between the two adjacent choline methylene groups at δ 3.69 and δ 4.34 (indicated in the figure with large black circles). With this information, the second methylene group can be assigned to the CH₂ at δ 62.1 (δ ¹³C) in the HSQC spectrum of blood plasma of FIG. 8A.

To get further insight into the nature of the phosphatidylcholine signals highlighted by STOCSY, titrations of various phosphatidylcholine standards into plasma were performed, as shown in the NMR plots of FIGS. 10A-10D. Chemical standards (1,2-dilinoleoyl-sn-glycero-3-phosphocholine, L-α-phosphatidylcholine, 1,2-dipalmitoyl-sn-glycero-3-phosphocholine and L-α-lysophosphatidylcholine) and α-1-acid-glycoprotein, were purchased from Sigma Aldrich (US). The plasma sample was removed from the magnet and 0.3-1.5 mg (depending on initial solubility) of the respective standard was directly added into solution. For α-1-acid-glycoprotein, 16 μL of a standard solution (9.3 mg of α-1-acid-glycoprotein in 70 μL 0.9% NaCl solution) was directly added into solution. The samples containing the standards were subjected to 30 minutes of sonication at ambient temperature (a test spectrum was acquired for this procedure without standard to ensure sonication did not disturb the plasma sample; data not shown). The samples were then inserted into the magnet and a second set of 1D experiments with water signal pre-saturation were acquired (using the DIRE sequence). FIG. 10A shows the results for 1,2-dilinoleoyl-sn-glycero-3-phosphocholine, FIG. 10B shows the results for L-α-phosphatidylcholine, FIG. 10C shows the results for 1,2-dipalmitoyl-sn-glycero-3-phosphocholine and FIG. 10D shows the results for L-α-lysophosphatidylcholine.

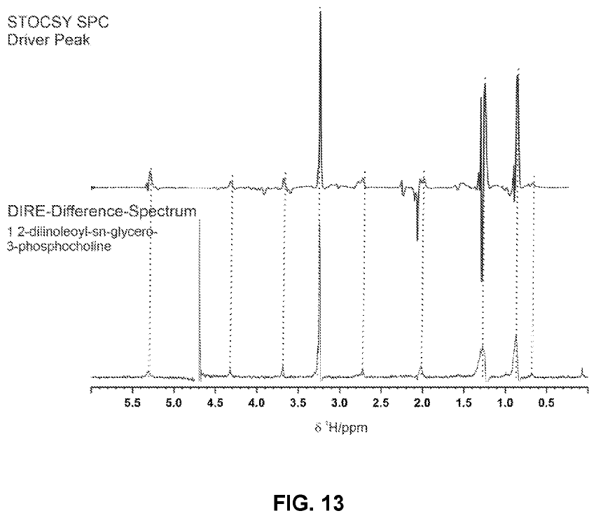

The NMR data were processed manually using Bruker Topspin™ 3.6.2 to achieve optimal phasing, baseline correction and spectral alignment. Difference spectra were calculated where the original (non-spiked) spectrum was subtracted from the spectrum containing the standard solution. All tested standards give rise to a similar signal pattern with an observable increase of SPC (SPC₁: δ 3.22 SPC₃: δ 3.26) as well as two signals at δ 3.69 and δ 4.34 corresponding to the methylene groups in the choline moiety, as shown in FIGS. 10A-10D. Additionally, signals at δ 0.8, 1.3, 2.0 and 5.3 increased, matching the respective alkyl-chain of the investigated standard. The L-α-lysophosphatidylcholine shows a high frequency shift of 16 Hz for the methylene group flanking the phospho-ester compared to the high-correlation spot highlighted by STOCSY, and is rather not the species that correlated with the selected pattern. Instead, the STOCSY signal pattern is more likely to indicate a correlation with a saturated phosphatidylcholine form with two aliphatic chains, such as dipalmitoyl-phosphatidylcholine, and it is likely that there are several saturated and unsaturated species present. The difference spectrum obtained by subtraction of the standard 1D NMR experiment before and after standard titration provides a clear spectrum of the standard with stoichiometric ratios for each signal. This suggests that all the added standards are bound and incorporated into the various lipoprotein particles restricting the molecular motions of the different phosphatidylcholine moieties. As the chemical shifts of the signals at δ 0.8 and δ 1.3 closely match the shifts observed in isolated HDL particles, it can be assumed that the detectable signals from the standards arise after incorporation into HDL particles. As all standards yield a similar spectral pattern which matches the STOCSY analysis, it can be assumed that SPCₜₒₜₐₗ presents a combination of manifold phosphatidylcholine species including saturated, unsaturated phosphocholine as well as their lyso-forms mainly located in HDL particles. A comparison of STOCSY and the DIRE-difference spectrum with 1,2-dilinoleoyl-sn-glycero-3-phosphocholine as standard is depicted in FIG. 13, and shows a perfect overlap of both spectra confirming that a significant portion of the signals in a DIRE spectrum come from phosphatidylcholines with a high proportion of unsaturated species.

Both the relative intensities of GlycA and GlycB and their sums give extremely good discrimination between Healthy and SARS-CoV-2 positive individuals with Kruskal-Wallis p-values in the 10⁻⁹ to 10⁻¹⁰ range (Table 6). The differences for healthy versus SARS-CoV-2 negatives and controls were significant, but much weaker, as were the differences between SARS-CoV-2 negatives and positives. This relation can also be observed in FIG. 6, which shows the GlycA and GlycB integrals for a healthy cohort, SARS-Cov-2 positive and SARS-COV-2 negative patients.

In FIG. 6, peak integrals determined using the DIRE spectra are shown for GlycA, which is shown to the left, and for GlycB, which is shown to the right. In the figure, the dots represent real data while the box plots are representations of the data distribution in terms of quantile. As shown, the data indicates that the relationship between GlycA and SPC changes between the SARS-CoV-2 positive patients and the healthy controls or SARS-CoV-2 negative participants. However, none of these parameters were significantly different between the healthy and SARS-CoV-2 negative patients (Table 6). The SPC composite peak also strongly distinguishes controls from SARS-CoV-2 positives (p=1.40×10⁻⁷) and, more importantly, between SARS-CoV-2 negative respiratory patients vs SARS-CoV-2 positives (p=4.52×10⁻⁸), indicating that this novel peak has differential diagnostic value. The SPC and GlycA have opposite signs and so the SPC: GlycA ratio was also highly significant for SARS-CoV-2 positives versus healthy or SARS-CoV-2 negatives, which makes this ratio a highly specific and sensitive diagnostic for SARS-CoV-2 induced metabolic phenoconversion.

The statistical relationships between the measured GlycA, GlycB and SPCₜₒₜₐₗ signals and the IVDR lipoprotein parameters derived from the same samples were investigated by the standard B.I.LISA method. There exist complex relationships between the lipoprotein patterns and other metabolic and cytokine data from COVID-19 patients, such as the inflammatory driven connections to COVID-19 dyslipidemia (elevated VLDL and LDL, and elevated Apolipoprotein A1/B100) and their possible implications in new onset diabetes and cardiovascular/atherosclerotic risk. Here, the lipoprotein data is used to establish a structural and compartmental connectivity to the novel SPCₜₒₜₐₗ data and the SPCₜₒₜₐₗ/Glycoprotein ratios. A strong pattern of correlation emerges between the SPCₜₒₜₐₗ and total plasma and total HDL Apolipoprotein A1 and A2 levels. This is because most of the plasma apolipoproteins are carried on HDL, which is significantly reduced in COVID-19. Similarly, there is a strong correlation between the SPCₜₒₜₐₗ signal and multiple HDL fraction concentrations, because the HDL phospholipids are in the same structural compartment as, for instance, the free cholesterol and total cholesterol. The exception is the weaker correlations with HDL-1 and HDL-2 fractions (phospholipids and cholesterol) because these are much less reduced in the disease. Thus, it may be inferred that a significant proportion of the SPCₜₒₜₐₗ component is present in the HDL subfraction 3 and HDL subfraction 4. There is also a correlation between the SPCₜₒₜₐₗ peak and the LDL-3, LDL-4, LDL-5 and LDL-6 peaks, but these are much weaker than the HDL correlations. So, on the basis of the diffusion edited STOCSY and the statistical IVDr correlations, one may conclude that the main composite SPCₜₒₜₐₗ diagnostic markers are from phospholipids in HDL-3 and HDL-4 with a contribution from the lysophosphatidylcholine (including a linoleoyl, 18:2 species) bound to α₁-1-acid glycoprotein, both of which are significantly lowered in COVID-19 disease. The fact that the α₁-1-acid glycoprotein is significantly elevated in COVID-19 as part of the inflammatory response makes the various ratios of GlycA/GlycB and SPC components particularly sensitive to the presence of the disease (Table 6).

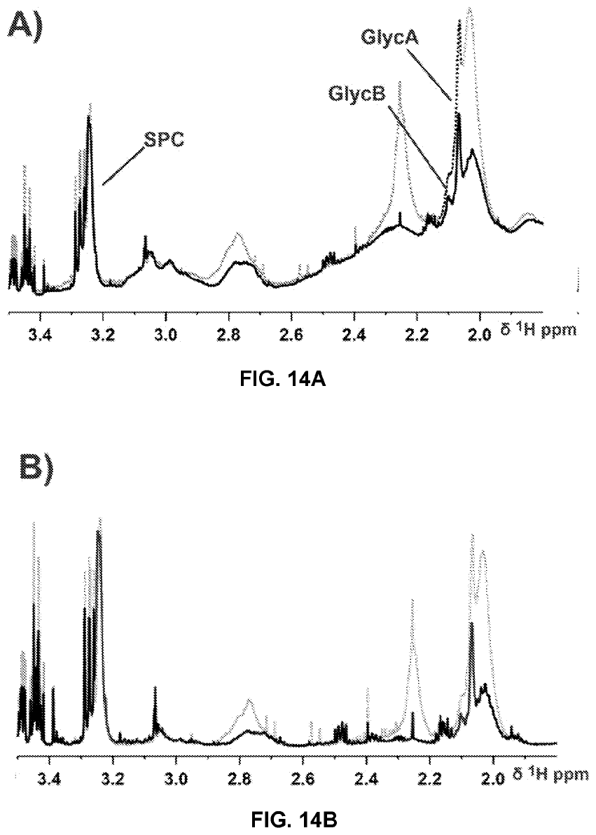

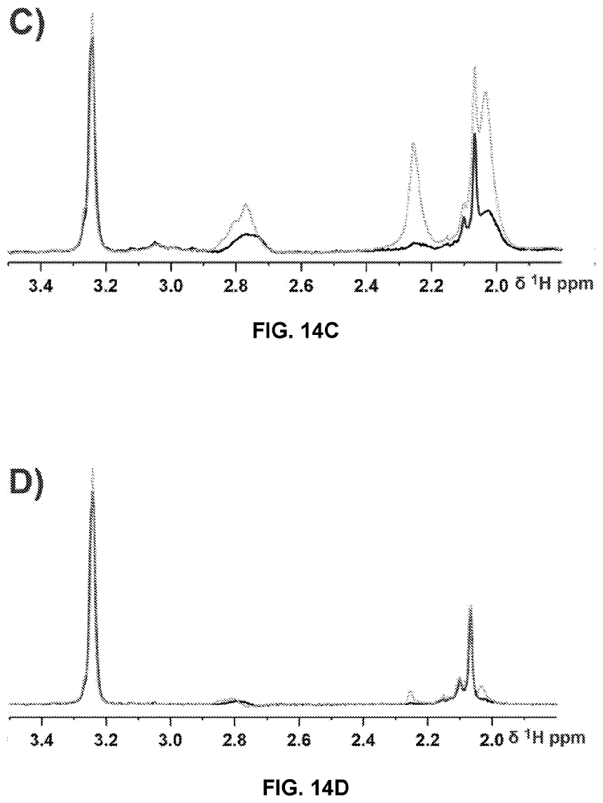

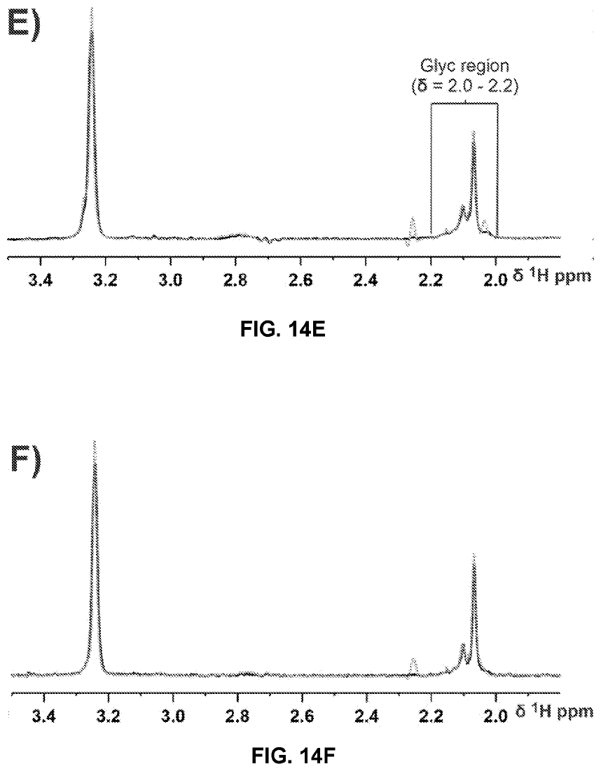

The results also indicate that JEDI provides inflammation marker quantification by simple peak integration. FIGS. 14A-14F provide a comparison of excerpts from a solvent suppressed proton spectrum, CPMG, DIRE, and JEDI approaches focussing on the spectral region of SPC and Glyc (GlycA+B). The spectra are presented for two serum samples, chosen to represent a typical spectrum (a medium serum lipoprotein concentration, shown in solid lines) and a severe outlier (a high serum lipoprotein concentration, shown in dashed lines), regarding the intensity of the overlapping lipid resonances at δ=˜2. In a regular ¹H NMR spectrum of serum, shown in FIG. 14A. SPC (3.25 ppm) and Glyc (2.07 and 2.1 ppm) are highly intricate showing interfering resonances from small molecules, lipoproteins and an overall protein background. The introduction of T₂ relaxation to filter out the broad protein envelope by applying a CPMG pulse train yields a flat baseline as a first editing step, the result of which is shown in FIG. 14B. DIRE adds another layer of editing by combining T₂ relaxation and diffusion to create a spectrum devoid of small molecule contributions and direct protein background, as shown in FIG. 14C, although some lipid peaks remained after both the relaxation and diffusion editing, which interfere with GlycA and GlycB. The DIRE experiment already allows for integration of SPC, but Glyc overlaps with the allylic-CH₂-stemming from lipoproteins, which are not significantly attenuated by the double filtering. However, the lipoprotein interference can be effectively attenuated by incorporating J-modulation in addition to T₂ relaxation and diffusion resulting in a triple edition experiment summarized as JEDI NMR. The J-modulation results in interference attenuation of the signal. FIG. 14D shows how the application of JEDI yields a clean spectrum that allows clear observation of the two biomarkers of interest. Whereas the —NMe₃⁺ of SPC and the N-acetyls of Glyc always get perfectly refocussed due to their absence of homonuclear J coupling, all other coupled systems get largely de-focussed. With JEDI, SPC and Glyc can simply be integrated, bypassing potential ambiguity from so far employed signal fitting methods.

It was also possible to perform an evaluation of the effectiveness of JEDI to filter lipoprotein contributions in various inflammatory states. Focussing on a serum sample with high levels of lipid/lipoprotein, shown as a gray dotted line in FIG. 14D, it is apparent that a residual lipoprotein signal is surviving the intensive spectral editing of JEDI. The spectrum of FIG. 14E demonstrates that JEDI-PGSE gives similar results to the PGPE but gives rise to increased antiphase contributions in the baseline and residual lipid peaks. The FIG. 14F spectrum demonstrates how JEDI-PGDE perfectly removes any lipids interfering with GlycA and GlycB but suffers from a lower signal-to-noise ratio.

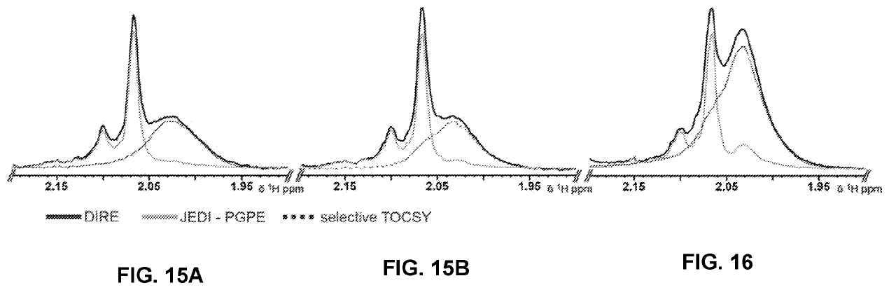

In another control experiment, the lipoprotein signal itself was investigated by selective TOCSY (TOtal Correlation SpectroscopY), the results of which are shown in FIGS. 15A-15B and 16. Each of these figures shows the results for a different serum sample, the samples of FIGS. 15A and 15B having medium lipoprotein concentration, while the sample of FIG. 16 has a high lipid concentration. The comparison of DIRE (solid black trace), JEDI (solid gray trace) and selective TOCSY (dotted trace) spectra showed that the JEDI and the selective TOCSY can reproduce the DIRE pattern further, indicating the absence of hidden interference signals below Glyc in the JEDI. Additionally, the selective TOCSY spectra show the high degree of heterogeneity of the lipoprotein response between the different serum samples of FIGS. 15A, 15B and 16. In fact, even similar DIRE spectra according to visual inspection (FIGS. 15A vs 15B) can have a quite different lipoprotein envelope. This is represented in a high frequency “shoulder” that perfectly overlaps with GlycA, which further skews results extracted using DIRE or fitting methods but is circumvented by using JEDI.

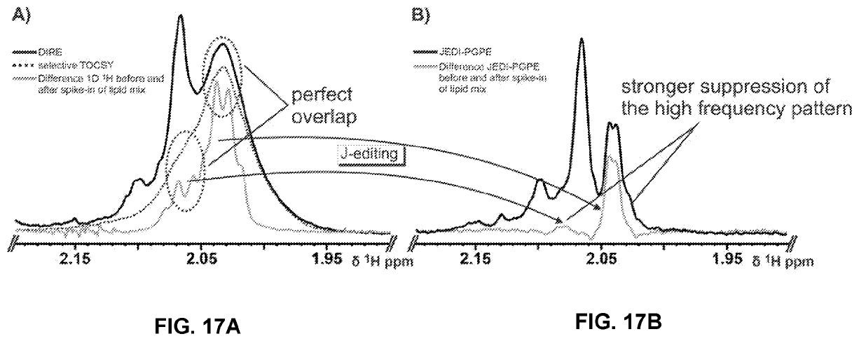

Comparing the lipoprotein signature obtained from the selective TOCSY spectra with the ones obtained from spiked lipid mixture spectra suggests that the high frequency shoulder of the lipoproteins is more attenuated than its low frequency counterpart as shown, for example, in FIGS. 17A-17B, which further enforces the robustness of the JEDI technique. FIG. 17A shows an overlap of a DIRE spectrum (black solid trace), the corresponding selective TOCSY (black dotted trace) and a difference spectrum (gray trace) before and after addition of a 2 μL lipid mix of parenteral nutrition yielding the net spectrum of the spiked-in lipids. FIG. 17B shows the overlap of the JEDI-PGPE before addition (solid black trace) and the JEDI-PGPE difference spectrum (solid gray trace) of a plasma sample before and after addition of a 2 μL lipid mix. Comparing the difference spectra from FIGS. 17A and 17B, it is evident that the JEDI experiment almost perfectly removes the high frequency “shoulder” by J-editing, explaining its high robustness for high lipid outliers. The net lipid pattern shows two distinct pairs of singlets (black dotted circles in FIG. 17A) suggesting the pattern of a quartet, where the high frequency quartet would have an apparent coupling constant of ˜6 Hz and the low frequency counterpart would have a coupling constant of ˜6.5 Hz. For comparison, the allylic —CH₂— lines of L-α-phosphatidylcholine show an apparent coupling constant of 7 Hz in CDCl₃ at 310K. The difference spectrum yields the net lipid mix pattern in solution. The net lipid pattern perfectly overlaps with the selective TOCSY for the high lipoprotein sample. Hence, the lipid mix is an acceptable surrogate to simulate high lipid concentration and the overlapping high frequency “shoulder” into Glyc. This improved suppression is possible due to a different coupling constant of the underlying resonances in the shoulder for the J-editing or due to different relaxation properties of the high and low frequency partitions.

The quartet pattern of the lipid signal is believed to be a pseudo-quartet and the result of an underlying doublet-of-triplets (dt) stemming from the coupling to the adjacent allylic proton attached to the sp₂ carbon (resulting in a doublet) and on the other end the adjacent —CH₂— (resulting in a triplet). The chemical shift of the two pseudo-quartets perfectly overlaps with the lipoprotein envelope of a high-concentration lipoprotein plasma sample (as shown by the black dotted circles in FIG. 17A). The resonance frequency of every lipoprotein moiety (here the allylic —CH₂—) directly correlates with the size of the particle (VLDL, IDL, LDL, or HDL), in which they are incorporated. Therefore, it can be assumed that the two pseudo-quartets of the net lipid pattern are the result of the lipid mixture being mainly incorporated in two different fractions and or subfractions; most likely VLDL as it naturally carries most of the triglycerides (main components of the lipid suspension). Application of the JEDI-PGPE significantly reduces the influence of the spiked-in lipid mixture. From the lipid pattern for the JEDI (shown in the difference spectrum of FIG. 17B of a JEDI-PGPE before and after addition of 2 μL lipid mix), it can be observed that the high frequency pseudo-quartet is significantly better suppressed than its low frequency counterpart. This behavior explains the robustness of the JEDI-PGPE for GlycA integration. The improved performance of the JEDI-PGPE for the suppression of the high frequency signal could be due to two reasons. First, the slightly different coupling constant for the two signals leads to a better dephasing during the J-editing of the high frequency signal and, second, according to its chemical shift the high frequency signal is incorporated in bigger, less-dense particles, which leads to a shorter T₂ relaxation time.

The optimal parameters for the JEDI-PGPE are chosen to find a compromise between acceptable signal-to-noise (S/N) ratio and effective removal of the interfering peaks. FIG. 4B depicts the DIRE sequence used in an exemplary embodiment of the invention, and is based on a stimulated echo (STE) experiment with an eddy current delay (bruker pulse program: ledbpgppr2s1d), whereas the delays in the first spin echo (tₛₑ) are significantly increased (16.6 ms) to allow for additional T₂ relaxation. Otherwise, the predominant relaxation pathway for the DIRE is T₁ relaxation. In order to defocus signals by J-editing, long evolution times in the xy-plane (dominated by T₂ relaxation, optimally 1/(2J) for the full spin echo; for the respective coupling) are required. Hence, the STE sequence is not ideal to incorporate J-editing as it would only lead to a needlessly long sequence with long T₁ and T₂ relaxation segments. In contrast, a less sophisticated sequence employing mainly T₂ relaxation (e.g., a conventional diffusion experiment Pulsed Gradient Spin Echo (PGSE)), which goes in line with the suggested ¹H s-filtered sequence is a more promising approach. FIG. 4F shows the employed JEDI sequence, the Pulsed Gradient Perfect Echo (PGPE), and highlights the main editing approaches of relaxation, diffusion and J-editing. In addition, JEDI-PGPE does not require any additional solvent suppression scheme when applied to serum and plasma samples.

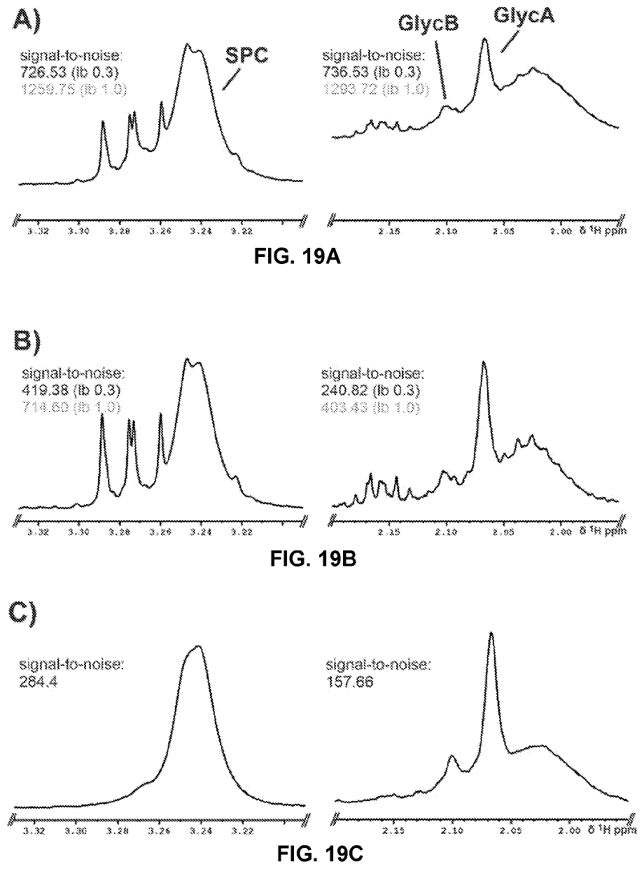

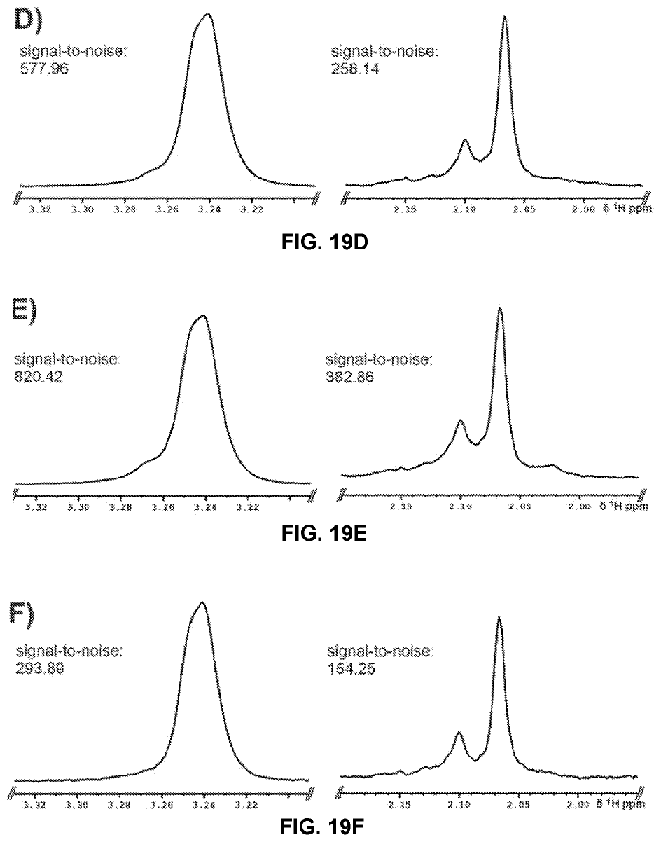

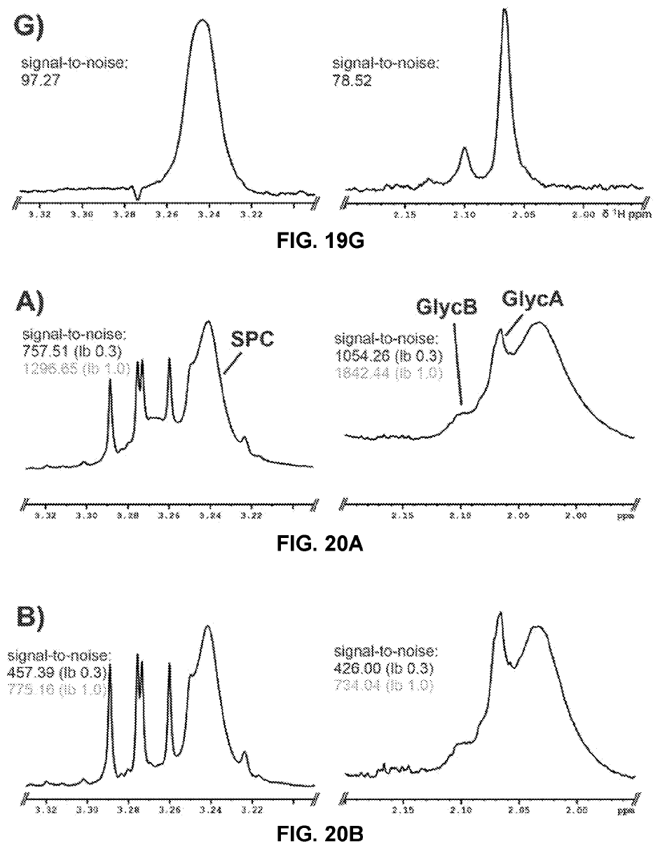

In addition to the JEDI-PGPE, three other related JEDI sequences were tested: the Pulsed Gradient Spin Echo (PGSE) (shown in FIG. 4G), the Pulsed Gradient Double Echo (PGDE) (shown in FIG. 4H) and the Pulsed Gradient Spin Echo×5 (PGSE-5) (shown in FIG. 4I). All sequences are based on one or multiple concatenated spin echoes employing varying refocusing delays. FIGS. 19A-19G each show proton spectra focussing on the SPC (left) and Glyc (right) region for sequences for a medium concentration lipoprotein serum sample, including the JEDI sequences mentioned above. The signal to noise for SPC (δ=3.17-3.33) and GlycA (δ=2.05-2.09) is given in the respective figure (noise region δ=−2.6−−5.2). The 1D with solvent suppression and the CPMG were processed with an exponential line broadening function of 0.3 Hz (according to the Bruker BioSpin Corp. IVDr method) and with a 1.0 Hz (grey writing) for better comparison with the DIRE and JEDI sequences. DIRE and the JEDI experiments were processed with an exponential line broadening function of 1.0 Hz.

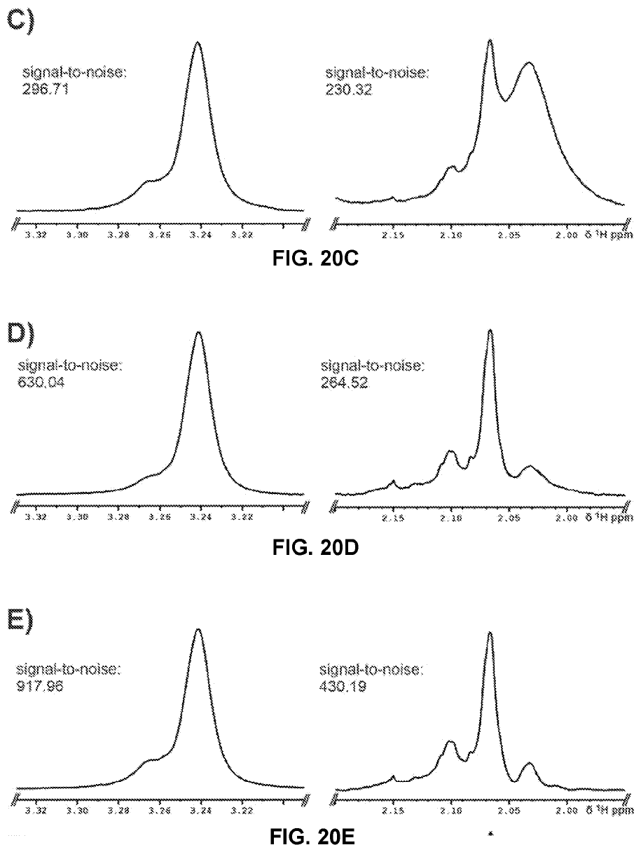

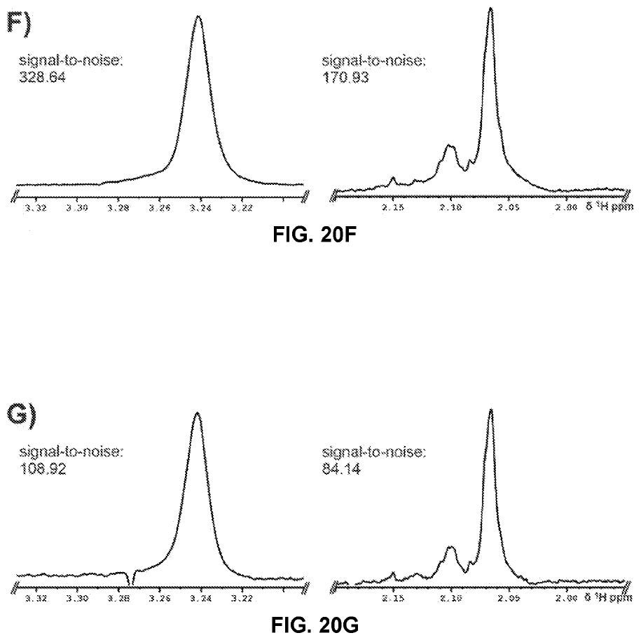

FIGS. 20A-20G each show proton spectra focussing on the SPC (left) and Glyc (right) region for sequences for a high concentration lipoprotein serum sample, including the JEDI sequences mentioned above. The signal-to-noise for SPC (δ=3.17-3.33) and GlycA (δ=2.05-2.09) is given in the respective figures (noise region δ=−2.6−−5.2). The 1D ¹H with solvent suppression and the CPMG were processed with an exponential line broadening function of 0.3 Hz (according to the Bruker IVDr method) and with a 1.0 Hz (grey text) for better comparison with the DIRE and JEDI sequences. DIRE and the JEDI experiments were processed with an exponential line broadening function of 1.0 Hz.

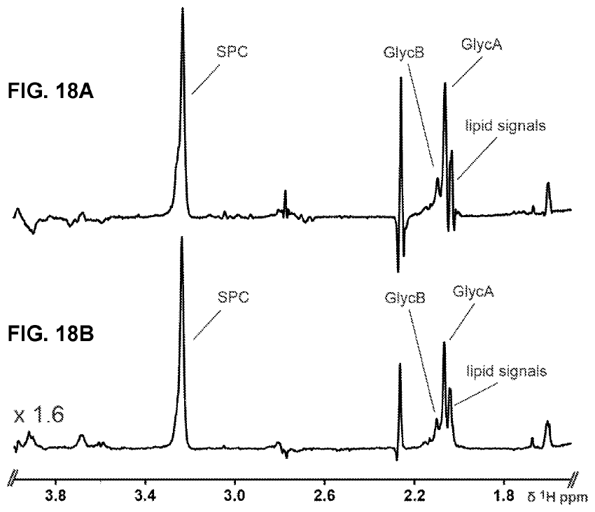

FIGS. 19A-19G and 20A-20G allow a comparison (at medium and high lipoprotein concentration, respectively) of the new JEDI sequences to the DIRE sequence for the two biomarkers SPC and Glyc (shown in FIGS. 19C and 20C). Results using the JEDI-PGPE sequence are shown in FIGS. 19D and 20D. Results using the new JEDI-PGSE sequence are shown in FIGS. 19E and 20E. FIGS. 19F and 20F show the results using the new JEDI-PGDE sequence. Finally FIGS. 19G and 20G show the results using the new JEDI-PGSE-5 sequence. The experimental parameters were adjusted so every experiment would have an approximate duration of 4.5 min as time is crucial in clinical laboratories. It is evident that JEDI sequences are capable of defocusing the overlapping lipoprotein resonances, but show significant differences in resulting signal intensity. The JEDI-PGSE and the JEDI-PGPE have by far the highest S/N ratio with effective suppression of interfering peaks. While its simplicity and high signal-to-noise ratio make JEDI-PGSE an ideal candidate, it leads to residual antiphase contributions, which are undesirable (as shown in FIGS. 18A and 18B). Both the JEDI-PGDE and JEDI-PGSE-5 perform well, but for low signal-to-noise ratio; especially for SPC-B. In summary, the JEDI-PGPE yielded excellent performance for applied criteria of high throughput NMR metabolomics, greatly outperforming the DIRE on Glyc determination and doubling the S/N ratio.

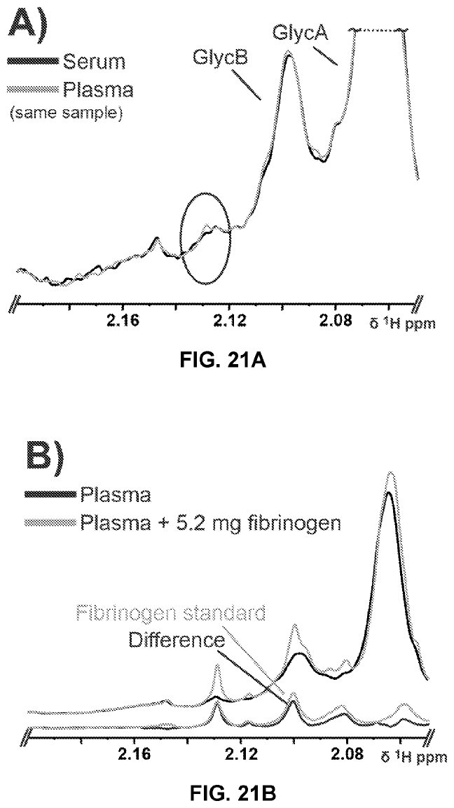

The results indicate that JEDI spectra of serum and plasma differ only by the contributions of fibrinogen. FIGS. 21A and 21B demonstrate that JEDI enables detection of fibrinogen. FIG. 21A provides a JEDI-PGPE comparison of the high frequency region of Glyc for a serum (black line) and plasma (gray line) sample of the same individual highlighting a small signal (black circle), which is present in plasma but absent in serum. As shown, the spectra look identical apart from a minor peak (δ=2.13) in the high frequency region of Glyc, which is present in plasma but absent in serum. A major difference between serum and plasma is the absence of clotting factors with fibrinogen as its main contributor. Therefore, a spike-in experiment with fibrinogen was performed, indeed identifying fibrinogen as an additional contributor to Glyc, as demonstrated in FIG. 21B, which shows a JEDI-PGPE high frequency region of Glyc for a plasma sample before (black line) and after (gray line) addition of 5.2 mg fibrinogen. Generating the difference spectrum (lower black line) shows good agreement with pure fibrinogen (lower gray line) and identifies the highlighted signal (black circle) in FIG. 21A as fibrinogen. As a clotting factor, fibrinogen is removed from serum samples explaining the observed difference between plasma and serum. It is therefore clear that the JEDI-PGPE gives new insights to the underlying nature of Glyc and reveals that Glyc has additional resonances at the higher frequency to the previously described regions of GlycA and GlycB.

The DIRE of FIG. 4B uses a combination of relaxation and diffusion to edit out contributions from large proteins (mostly albumin; broad background in NMR spectrum) and small molecules to yield a subset of species, which are associated to high molecular weight structures (low diffusion coefficient) but still retain a high degree of mobility (long T₂ times). Afterwards, the magnetization is placed along the z-axis during the remaining diffusion scheme to minimize relaxation losses. For the determination of SPC and Glyc, DIRE provides an acceptable signal-to-noise as well as a flat baseline but suffers from the drawback that it barely suppresses lipoprotein resonances, which inter alia overlap with Glyc.

The general idea of JEDI is to combine T₂ relaxation, diffusion and J-editing in one compact sequence and tailor the parameters for the quantification of SPC and Glyc while also retaining a high signal-to-noise ratio for application in high throughput serum and plasma analysis. Here, T₂ relaxation has to be sufficient/long enough to allow for relaxation of the broad protein signal background in a serum or plasma spectrum and diffusion has to be adjusted to remove all small molecule contributions. Among the plethora of NMR editing approaches, suppression of coupled spin systems is achieved by spin-echoes with evolution delays tailored towards the signal (J-editing) which is to be suppressed. For perfect suppression of a coupling constant by J-editing, one would adjust the total duration of a spin echo to 1/(2J), when the signal yields perfect antiphase magnetization. Although this theoretical background was taken into consideration (coupling constants for the respective lines in question from standards, like l-α-phosphatidylcholine in CDCl₃ at 310K were determined to be ˜7 Hz resulting in 1/(2J)=˜71 ms), spin echo delays still had to be determined experimentally, because the 1/(2J) relation only holds true for one specific coupling constant at a time and the overlapping lipoprotein of Glyc constitutes an array of different signals with different coupling constants. Additionally, an attempt was made to minimize relaxation losses, opting for short durations of the spin echoes.

In summary, a JEDI sequence should contain a diffusion scheme and one or more spin echo blocks, and the magnetization must be stored in the xy-plane for a sufficient amount of time to allow T₂ relaxation. FIG. 4F depicts the Pulsed Gradient Perfect Echo (PGPE) and highlights the three employed editing techniques of relaxation, diffusion and J-editing in the sequence. The sequence is similar to the previously published J-compensated PGSE, and the known “PROJECTED” sequence, which uses a perfect echo block with gradients, but incorporates an additional z-filter and uses the spin-echo delays (tₛₑ) for J-editing instead of improved refocussing. This is counterintuitive, because improved refocusing of homonuclear J-couplings was the intended use of the perfect echo block. T₂ is the main relaxation pathway throughout the whole sequence apart from the quick storage of the magnetization in the z-axis during the z-filter. With tse set to 27.5 ms, the total T₂ relaxation time accumulates to 110 ms. This value was determined experimentally. Longer tse delays result in slightly better suppression of the lipoprotein signal but affect signal intensity negatively, whereas for shorter tse delays lipoprotein suppression becomes unacceptable (for outliers). The JEDI-PGPE yields good signal-to-noise (as shown below in Table 7), and has a flat baseline and effectively suppresses the overlapping lipoprotein signal. The nature of the baseline and the residual lipoprotein signals is one of the main differences between the JEDI-PGPE and JEDI-PGSE, because all residuals of the JEDI-PGPE are mostly in phase, whereas for the JEDI-PGSE all residual signal have strong anti-phase contributions. This is demonstrated in FIGS. 18A and 18B which show, respectively, a JEDI-PGSE and a JEDI-PGPE for a serum sample spiked with 2 μL of lipid mixture from parenteral nutrition (˜80% olive oil, ˜20% soybean oil). Although the JEDI-PGSE of FIG. 18A has a better signal-to-noise (˜1.6; spectra were scaled for better visibility), it is evident that the JEDI-PGSE has baseline issues as the residual lipoprotein signals have a strong antiphase contribution, which also interferes with the Glyc composite. In contrast, the JEDI-PGPE provides a mostly flat baseline even for extreme outliers.

The Pulsed Gradient Spin Echo (PGSE) (FIG. 4G) represents the simplest of all JEDI-sequences. As the classic diffusion sequence, it employs a single spin echo flanked by two diffusion gradients followed by a quick z-filter. Tₛₑ was determined experimentally and the best trade-off between signal-to-noise and lipoprotein suppression was found for a value of 30 ms. The PGSE yields the highest signal-to-noise of all JEDI sequences (for SPC and Glyc) and has an acceptable lipoprotein suppression (Table 7). The issues of the PGSE are found in the nature of the residual signals, which contain strong anti-phase contributions, as mentioned above with regard to FIGS. 18A and 18B. For analysis, this resulted in unreliable GlycA integration results for strong lipoprotein outliers and spectral phasing was unreliable in automation and had to be manually corrected.

The Pulsed Gradient Double Echo (PGDE) (FIG. 4H) is an elaboration of the PGSE containing a second spin echo block. A second spin echo allows for a better coverage of suppressed coupling constants. The tseland tₛₑ₂ delays were determined experimentally at 37.5 (tₛₑ₁) and 60 ms (tₛₑ₂) with the aim of maximum suppression of the overlapping lipoprotein resonances. Only long relaxation delays gave a full suppression of the lipoproteins. Shorter delays give worse suppression and longer delays can lead to a re-appearance of the lipoprotein signals with negative phase (data not shown). After each spin echo, a z-filter is placed to further clean up the spectrum. The PGDE gives excellent suppression of the overlapping lipoprotein resonances of Glyc but due to the long tse delays it suffers from strong signal-to-noise losses.

The Pulse Gradient Spin Echo×5 (PGSE-5) (FIG. 4I) is the recreation of the sequence postulated by Martin-Pastor for ¹H s-filtered experiments but tuned for the JEDI application, which is the incorporation of a gradient scheme to remove small molecule contributions. The sequence is a series of five concatenated spin-echoes, each employing an incremental spin-echo delay (tₛₑ₁₋₅) to cover a large J-coupling range. After each spin echo, a z-filter removes residual signals in the xy-plane. The original sequence also included the adiabatic M. Thrippleton and J. Keeler z-filter, but it was removed as it did not improve spectral quality and led only to longer relaxation. Two pulsed gradient schemes employed during the first and last spin-echo were found to be sufficient to remove small molecule contributions. The PGSE-5 does a perfect job at dephasing coupled spin systems and thoroughly eliminates lipoprotein interferences from Glyc (as shown in FIGS. 19G and 20G). However, the accumulated spin-echo time in the PGSE-5 amounts to ˜400 ms, resulting in a low signal-to-noise ratio for Glyc and SPC; in particular, the contributions from SPC-B (the high frequency part of SPC stemming from LDL phospholipids) are almost entirely removed (as shown in FIGS. 19G and 20G).

Another trade-off had to be made for the interscan delay (relaxation delay+acquisition time). For the determination of interscan delay, JEDI contains three signals of interest. First, SPC; second, Glyc and third, the residual lipoprotein signal, which can interfere with Glyc. Rough estimation of the T₁ relaxation times by inversion recovery experiments yielded the following result for T₁ times: SPC<lipoprotein<Glyc. This means that for short interscan delays (˜2 s) signal/time is well suited for SPC but it leads to stronger interferences of the lipoproteins with Glyc because the lipoproteins are quicker to return to equilibrium. On the other hand, for long interscan delays (˜7 s) Glyc is more interference-free but signal-to-noise is “wasted” for SPC. Hence, the interscan delay was determined experimentally with a value of ˜4 s evaluating overall signal-to-noise against lipoprotein interference of Glyc. Other sequences which use J-editing like the HAL and the SQF experiment postulated by Kojima et al. were also modified (diffusion) and tested, but did not yield better (or acceptable) results compared to the JEDI sequences described above.

Signal-to-noise performance for SPC (δ=3.17-3.33) and GlycA (δ=2.05−2.09) regions evaluated for different sequences including IVDr methods, DIRE and the JEDI approach. Signal-to-noise was extracted with the help of TOPSPIN by the use of the.sino (noise region δ=−2.6−−5.2) application.

TABLE 7
Comparison of S/N for different pulse sequences

| Sample | Signal | 1D ¹H | CPMG | DIRE | PGPE | PGSE | PGDE | PGSE-5 |
|---|---|---|---|---|---|---|---|---|
| 1 | SPC | 726.5 | 419.4 | 284.4 | 578.0 | 820.4 | 293.9 | 97.3 |
| | GlycA | 736.5 | 240.8 | 157.7 | 256.1 | 382.9 | 154.3 | 78.5 |
| 2 | SPC | 757.5 | 457.3 | 296.7 | 630.0 | 918.0 | 328.6 | 108.9 |
| | GlycA | 1054.3 | 426.0 | 230.3 | 264.5 | 430.2 | 170.9 | 84.1 |
| 3 | SPC | 494.3 | 301.0 | 99.6 | 204.0 | 291.5 | 111.9 | 30.5 |
| | GlycA | 863.0 | 319.6 | 205.3 | 324.6 | 487.4 | 218.2 | 103.8 |

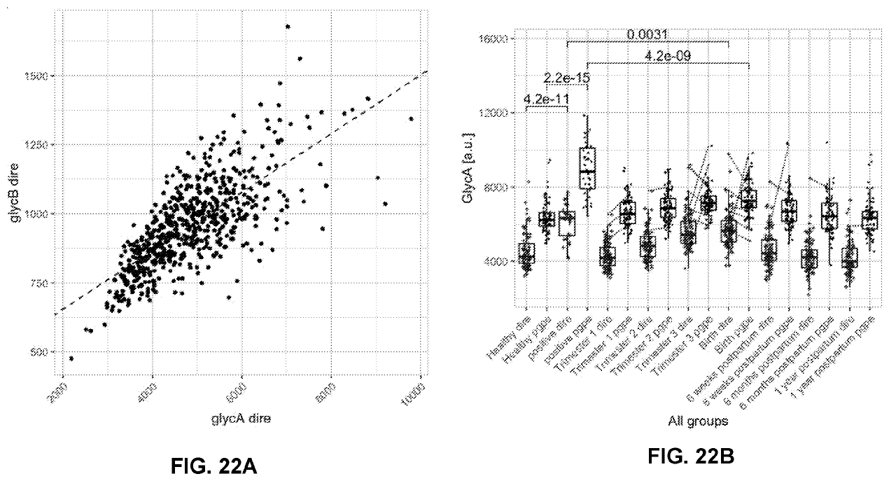

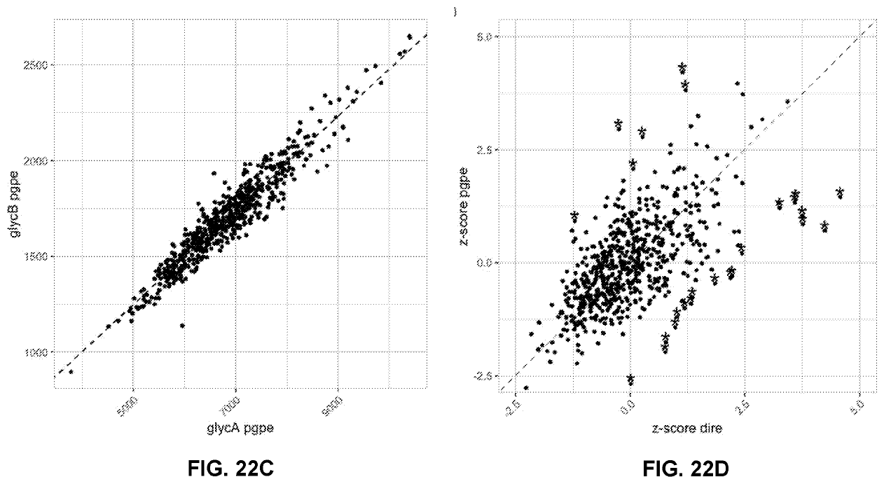

FIGS. 22A-22D evidence the improvement in accuracy that is provided by JEDI. The statistical analyses compare JEDI and DIRE for a dataset of 631 longitudinally collected serum samples of pregnant women and a second dataset containing 116 samples of healthy (n=80) and SARS-CoV-2(+) (n=36) specimens. FIG. 22A shows only a weak correlation between GlycA and GlycB intensities due to the DIRE data being scrambled by the overlapping lipoprotein peaks. In contrast to DIRE, the JEDI-PGPE reveals a high correlation between GlycA and B (FIG. 22B) indicating that despite their heterogeneous nature the ratio of underlying glycoproteins remains fairly constant. FIG. 22C demonstrates for a real cohort how DIRE may lead to erroneous results in presence of strong overlapping signal from lipoproteins. Z-scores (FIG. 22D) indicates which samples changed their respective rank (“mis-classification”) for DIRE against JEDI. The strongest outliers are marked with an asterisk.

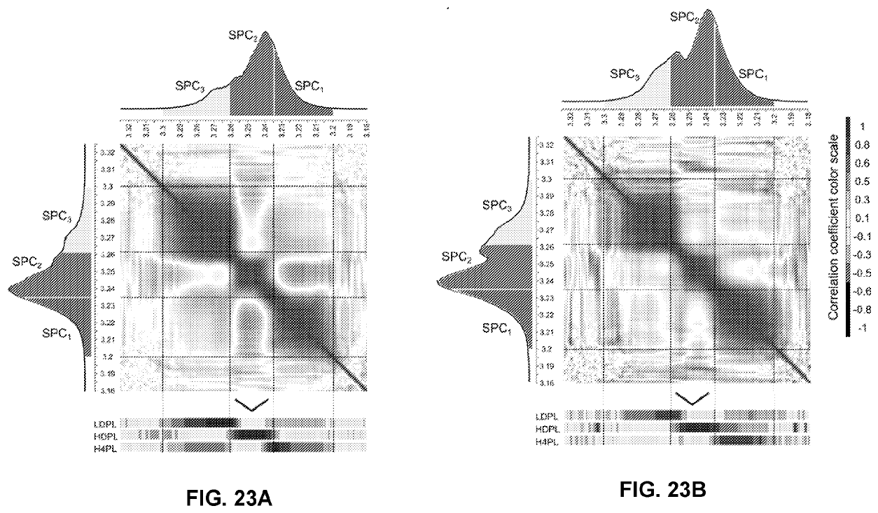

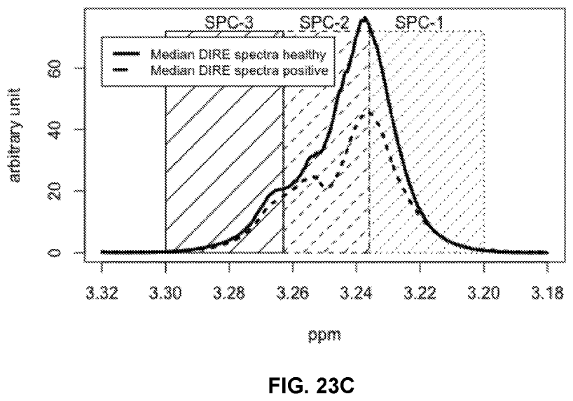

Next to the sub-splitting of the SPC region (δ=3.17-3.33) into a low-frequency (SPC-A) and high-frequency (SPC-B) region according to the main fractions of HDL (SPC-A) and LDL (SPC-B), it is also possible to further subdivide the SPC region by correlation analysis. This is demonstrated by the control and SARS-CoV-2 positive heatmaps shown, respectively, in FIGS. 23A and 23B. The heatmaps are of the SPC spectral region using DIRE NMR experiments. Three distinct SPC spectral high-correlation regions are observed with chemical shift boundaries optimised using Spearman's rs (range 0.6-1.0), and are identified as SPC₁, SPC₂ and SPC₃. In addition, correlation of SPC₁, SPC₂ and SPC₃ to the IVDr lipoprotein parameters total LDL-Phospholipids (LDPL), total HDL-Phospholipids (HDPL) and Phospholipids of the highest density HDL subfraction (H4PL) are depicted in the lower portion of each figure, and show strong correlations of SPC₃ to LDPL, SPC₂ to HDPL and SPC₁ to H4PL, respectively, resulting in a more detailed subdivision of the SPC region. Hence, the notation of the SPC region into SPC₁, SPC₂ and SPC₃ improves on the notation of just SPC-A and SPC-B by yielding additional information about lipoprotein subfractions and providing a more exact frequency cut-off for integration. These SPC subregions are also shown in the standalone plot of FIG. 23C, which depicts the median DIRE spectra for samples from healthy patients (solid line trace) and SARS-CoV-2 infected patients (dotted line trace), respectively.

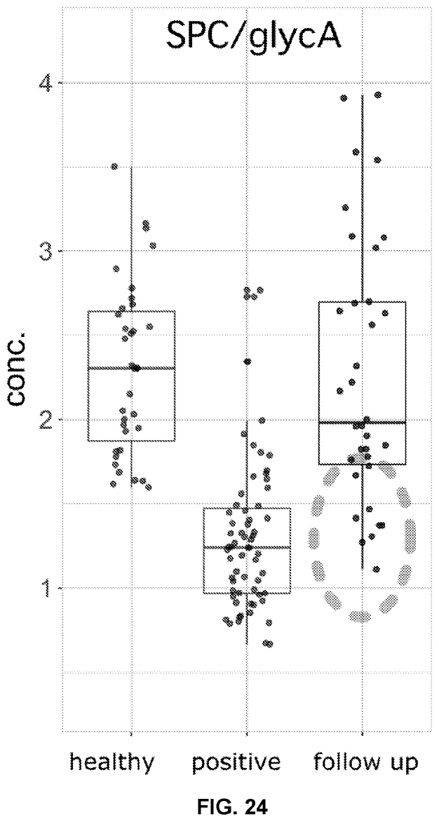

The SPC/Glyc ratio showed to be a useful marker for disease recovery. FIG. 24 plots the SPC/Glyc ratio for a control cohort, a SARS-CoV-2 positive cohort and the same cohort 3 months after the acute phase, and illustrates the inhomogeneous normalization process, phenoreversion, after infection by SARS-CoV-2 (known as phenoconversion). The ratio SPC/GlycA is back to normal after 3 months for most patients, while some of them are still found outside of the expected range for a normal population (highlighted by the dashed gray ellipse). Follow up samples (n=27; one patient has multiple time points) were collected at 3 months in Western Australia and modelled against normal controls (healthy group; n=41) and infected peoples (positive group; n=18 with multiple time points). Evidently, according to the applied ratio of SPC and Glyc, most people have recovered after 6 months, whereas a portion of the specimen still shows an abnormal SPC/Glyc ratio indication that some people are still not metabolically recovered even after 6 months.

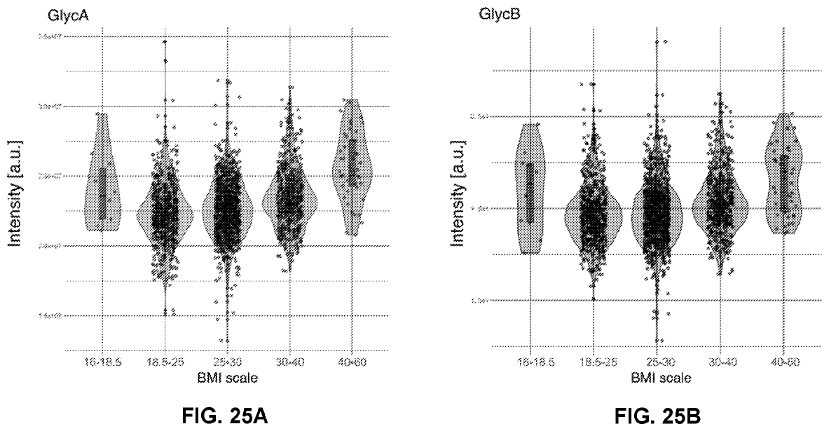

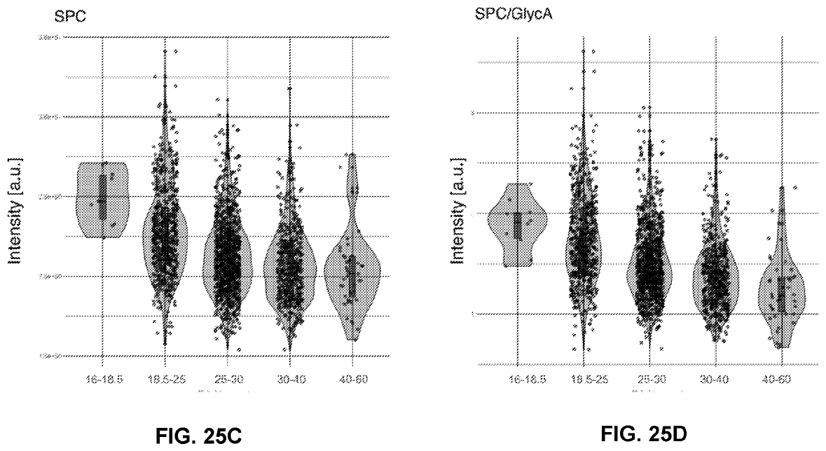

It has been shown that SPC (and its sub-regions), Glyc (and its sub-regions) and the SPC/Glyc ratio are powerful markers to assess acute inflammation during the acute phase of COVID infection and they can also be used to interrogate recovery after the acute phase of COVID. Furthermore, the markers SPC (and its sub-regions), Glyc (and its sub-regions) and the SPC/Glyc ratio can be employed as general markers of inflammation, e.g., chronic inflammation, not just limited to COVID. FIGS. 25A-25D show the integrals of GlycA, GlycB, SPC and the SPC/GlycA ratio from a large, normal population cohort (n=2045) stratified into BMI intervals ranging from 16-60, and indicates how the Glyc and SPC biomarkers capture chronic inflammation. The BMI intervals used were 16-18.5 (n=10); 18.5-25 (n=573); 25-30 (n=912); 30-40 (n=506); and 40-60 (n=44). SPC and SPC/GlycA ratio are shown capturing a downward trend that is not captured by Glyc only markers (GlycA and GlycB), thereby highlighting the potential of SPC for a broader range of application than COVID-19 and its complementarity with Glyc markers. GlycA and GlycB show a slight upwards trend from the nominally ideal BMI range (18.5-25) towards higher BMI ranges indicating increased inflammation with higher BMI values. The data also suggests a higher GlycA and GlycB for the low BMI range of 16-18.5, but this could also be skewed due to the low n (<10) and requires further investigation. The downward trend shown by SPC extends throughout all BMI ranges from 16 to 60, suggesting increased inflammation at higher BMI indices. Accordingly, the SPC/GlycA ratio shows a downwards trend throughout the BMI ranges as well, thereby highlighting the potential of SPC for a broader range of application than COVID-19.

As described herein, a combination of diffusion, relaxation and J-coupling edited NMR spectroscopy of blood plasma provides excellent discrimination of SARS-CoV-2 positivity from controls or SARS-CoV-2 negative patients based on the enhanced detection of occult diagnostic compartments. The same strategy yields excellent markers for inflammation (GlycA and GlycB) and cardiovascular risk (SPC₃/SPC₂ ratio). The key diagnostic species are from the total composite NMR signals (the supramolecular phospholipid composite, SPC) from terminal head groups in phospholipids, together with (lyso-)phosphatidylcholine and sphingomyelin, from HDL and LDL and the glycoprotein N-acetyl composite signal Glyc. Glyc has several distinguishable components, currently assigned as GlycA, GlycB (main contributions) and other glycoproteins such as fibrinogen (in lesser amounts). SPC has several distinguishable components—currently described as SPC₁, SPC₂ and SPC₃—that are associated to the different fractions and subfractions: these carry different but related diagnostic information. These markers appear to offer excellent diagnostic discrimination, and the high speed of the DIRE/JEDI (or the like, PGSE, PGDE, PGPE) experiments could be exploited as a direct phenoconversion test to help augment conventional PCR results, which could be of value in biosecurity situations. For many COVID-19 patients, recovery is slow or incomplete, and SPC/Glyc ratios could also be employed as measures of functional systemic recovery. SPC₃/SPC₂ ratio could be used to evaluate the shift in cardiovascular risk infected people are exposed to. The DIRE/JEDI diagnostic is unusual in that it utilises the dynamic motional properties of the biomarker molecules as well as concentration variations to enhance classification of the disease over methods employing simple concentration metrics, and thus represents a new class of molecular dynamic diagnostic. These data also illustrate well how untargeted NMR spectroscopic exploration can be readily translated into targeted measurements that can be performed on the same sample within the same experiment.

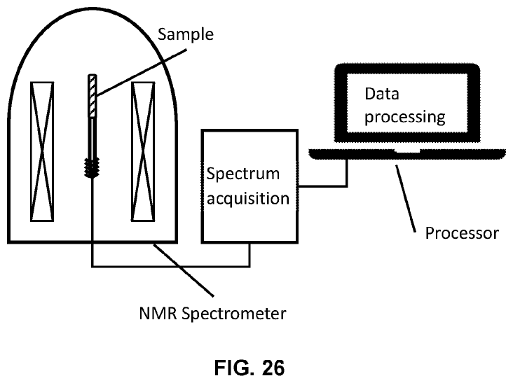

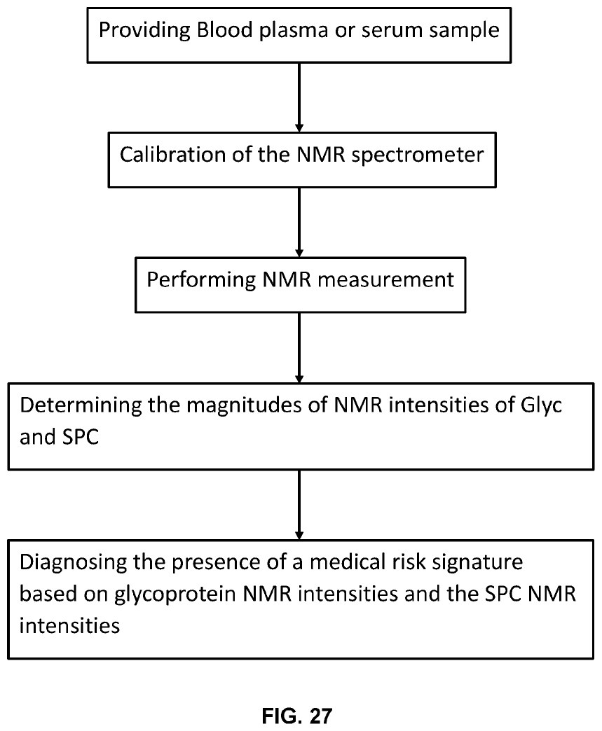

A system that may be used for performing the necessary measurements and diagnosis is shown in FIG. 26. An NMR spectrometer is used to measure an in vitro sample from a patient, and the measurements are provided to a data processor that is programmed to perform an analysis thereof according to the invention. Given the proper parameters, the system can therefore provide a diagnosis of a particular medical risk condition being investigated. The general diagnostic method being followed is depicted in the flow diagram of FIG. 27. As shown, an appropriate sample is provided, and the NMR spectrometer is calibrated as necessary. The NMR measurement is then performed and the magnitudes of the NMR intensities of the Glc and SPC signals are obtained. Based on the molecular markers discussed above that are demonstrated by those signals, the medical risk signature in question is then diagnosed.

1. A 1H-NMR spectroscopic molecular marker for identifying a medical risk signature in an in vitro blood plasma or serum sample from a patient by 1H NMR spectroscopy, wherein the marker comprises a combination of NMR intensity signals having magnitudes that are significantly different from known corresponding NMR intensity levels for a healthy patient, including a Glyc signal from at least one N-acetyl (—NCOCH₃) glycoprotein and an SPC signal from a choline head group (⁺N—(CH₃)₃) of a supramolecular phospholipids cluster (SPC) present in HDL and LDL lipoprotein subfractions.

2. A molecular marker according to claim 1, wherein the Glyc signal is in a chemical shift region from δ=2.00 ppm to δ=2.20 ppm and the SPC signal is in a chemical shift region from δ=3.20 ppm to δ=3.30 ppm.

3. A molecular marker according to claim 1, wherein the Glyc signal comprises a plurality of NMR intensity signals, including a signal GlycA in a chemical shift subregion of δ=2.00 ppm to δ=2.09 ppm and a signal GlycB in a chemical shift subregion of δ=2.09 ppm to δ=2.2 ppm.

4. A molecular marker according to claim 1, wherein the SPC signal comprises a plurality of NMR intensity signals including a signal SPC₁ in a chemical shift subregion from δ=3.2 ppm to δ=3.235 ppm, a signal SPC₂ in a chemical shift subregion from δ=3.235 ppm to 3.26 ppm, and a signal SPC₃ in a chemical shift subregion from δ=3.26 ppm to δ=3.3 ppm.

5. A molecular marker according to claim 4, wherein the molecular marker further comprises a ratio of NMR peak intensities of Glyc or either of GlycA or GlycB to NMR peak intensities of SPC or one or more of SPC₁, SPC₂ or SPC₃.

6. A molecular marker according to claim 1 wherein the medical risk signature comprises a SARS-CoV-2 infection of the patient.

7. Use of the molecular marker of claim 6 for the diagnosis of SARS-CoV-2.

8. A molecular marker according to claim 1 wherein the medical risk signature comprises acute inflammation.

9. Use of the molecular marker of claim 8 for the diagnosis of acute inflammation.

10. A molecular marker according to claim 1 wherein the medical risk signature comprises a known cardiovascular risk condition.

11. Use of the molecular marker of claim 10 for the diagnosis of said known cardiovascular risk condition.

12. Use of the molecular marker of claim 6 for the assessment of functional recovery from a SARS-CoV-2 infection.

13. A method of diagnosing a medical risk signature in a patient, the method comprising:
obtaining a blood plasma or serum sample from the patient;
performing an NMR measurement of the sample to obtain a spectrum of NMR intensities;
determining the magnitudes of a combination of NMR intensity signals, including a Glyc signal from at least one N-acetyl (—NCOCH₃) glycoprotein and an SPC signal from a choline head group (⁺N—(CH₃)₃) of a supramolecular phospholipids cluster (SPC) present in HDL and LDL lipoprotein; and
diagnosing the presence of said medical risk signature when the magnitudes of the glycoprotein NMR intensities and the SPC NMR intensities are significantly different from known corresponding NMR intensity levels for a healthy patient.

14. A method according to claim 13 wherein the Glyc signal is in a chemical shift range from δ=2.00 to δ=2.20 ppm and the SPC signal is in a chemical shift range from δ=3.20 to δ=3.30 ppm.

15. A method according to claim 13, wherein the Glyc signal comprises a plurality of NMR intensity signals, including a signal GlycA in a chemical shift subregion of δ=2.00 ppm to δ=2.09 ppm and a signal GlycB in a chemical shift subregion of δ=2.09 ppm to δ=2.2 ppm.

16. A method according to claim 13, wherein the SPC signal comprises a plurality of NMR intensity signals including a signal SPC₁ in a chemical shift subregion from δ=3.2 ppm to δ=3.235 ppm, a signal SPC₂ in a chemical shift subregion from δ=3.235 ppm to 3.26 ppm, and a signal SPC₃ in a chemical shift subregion from δ=3.26 ppm to δ=3.3 ppm.

17. A method according to claim 16, further comprising determining a ratio of NMR peak intensities of at least one of Glyc, GlycA and GlycB to NMR peak intensities of at least one of SPC, SPC₁, SPC₂ or SPC₃ and diagnosing the presence of said medical risk signature when the magnitude of the ratio is significantly different from a known corresponding ratio for a healthy patient.

18. A method according to claim 16 wherein the method further comprises calculating a ratio SPCₜₒₜₐₗ/GlycA, where SPCₜₒₜₐₗ=SPC₁+SPC₂+SPC₃, and diagnosing the presence of said medical risk signature when the magnitude of the SPCₜₒₜₐₗ/GlycA ratio is past a predetermined threshold.

19. A method according to claim 16 wherein the method further comprises calculating a ratio of any one of the signals SPC₁, SPC₂ or SPC₃ to the GlycA signal and diagnosing said medical risk signature when the ratio is beyond a predetermined threshold.

20. A method according to claim 13 further comprising diagnosing the presence of said medical risk signature when the Glyc signal intensity is significantly elevated and the SPC signal intensity is significantly reduced relative to said known corresponding NMR signal intensity levels for a healthy patient.

21. A method according to claim 13 wherein the medical risk signature comprises a SARS-CoV-2 infection of the patient.

22. A method according to claim 13 wherein the medical risk signature comprises acute inflammation.

23. A method according to claim 13 wherein the medical risk signature comprises a known cardiovascular risk condition.

24. A method according to claim 13 wherein the NMR measurement is performed with an NMR instrument that uses a DIffusional and Relaxation Editing (DIRE) pulse sequence or a J-coupling edited (JEDI) DIRE pulse sequence.

25. A system for identifying a medical risk signature using an in vitro blood plasma or serum sample from a patient, the system comprising:
an NMR spectrometer for acquiring at least one 1H NMR spectrum of the vitro blood plasma or serum sample; and
a data processor in communication with the NMR spectrometer, the data processor configured to obtain concentration measurements of an NMR spectroscopic molecular marker comprising a combination of NMR intensity signals having magnitudes that are significantly different from known corresponding NMR intensity levels for a healthy patient, including a Glyc signal from at least one N-acetyl (—NCOCH₃) glycoprotein and an SPC signal from a choline head group (⁺N—(CH₃)₃) of a supramolecular phospholipids cluster (SPC) present in HDL and LDL lipoprotein subfractions.

26. A system according to claim 25 wherein the Glyc signal is in a chemical shift region from δ=2.00 ppm to δ=2.20 ppm and the SPC signal is in a chemical shift region from δ=3.20 ppm to δ=3.30 ppm.

27. A system according to claim 25 wherein the Glyc signal comprises a plurality of NMR intensity signals, including a signal GlycA in a chemical shift subregion of δ=2.00 ppm to δ=2.09 ppm and a signal GlycB in a chemical shift subregion of δ=2.09 ppm to δ=2.2 ppm.

28. A system according to claim 25 wherein the SPC signal comprises a plurality of NMR intensity signals including a signal SPC₁ in a chemical shift subregion from δ=3.2 ppm to δ=3.235 ppm, a signal SPC₂ in a chemical shift subregion from δ=3.235 ppm to 3.26 ppm, and a signal SPC₃ in a chemical shift subregion from δ=3.26 ppm to δ=3.3 ppm.

29. A system according to claim 28 wherein the molecular marker further comprises a ratio of NMR peak intensities of Glyc or either of GlycA or GlyCB to NMR peak intensities of SPC or one or more of SPC₁, SPC₂ or SPC₃.

30. A system according to claim 25 wherein the medical risk signature comprises a SARS-CoV-2 infection of the patient.

31. A system according to claim 25 wherein the medical risk signature comprises acute inflammation.

32. A system according to claim 25 wherein the medical risk signature comprises a known cardiovascular risk condition.
